(19) United States
(12) Patent Application Publication
(10) Pub. No.: US 2010/0266532 A1
(43) Pub. Date: Oct. 21, 2010

(54) METHODS FOR INHIBITING SCARRING

(75) Inventor: Mark William James FERGUSON, Manchester (GB)

Correspondence Address: FOLEY AND LARDNER LLP;SUITE 500, 3000 K STREET NW, WASHINGTON, DC, 20007, US

(73) Assignee: Renovo Limited

(21) Appl. No.: 12/747,656

(22) PCT Filed: Dec. 11, 2008
(86) PCT No.: PCT/GB08/04081

§ 371 Date: Jun. 11, 2010

(30) Foreign Application Priority Data
Dec. 12, 2007 (GB) 0724231.6

(51) Int. Cl. A61K 38/20; C07K 14/54; A61P 17/02; B65D 71/00

(52) U.S. Cl. 424/85.2; 530/351; 206/223

(57) ABSTRACT

The invention provides new methods of treatment using anti-scarring agents to inhibit scarring in humans, and also provides anti-scarring agents for new uses in the inhibition of scarring in humans. In a first incidence of treatment an anti-scarring agent is provided to each centimetre of a wound margin or each centimetre of a site at which a wound is to be formed in a first therapeutically effective amount; and in a subsequent incidence of treatment the anti-scarring agent is provided to each centimetre of wound margin in a larger therapeutically effective amount. The incidences of treatment occur between 8 hours and 48 hours apart from one another. The anti-scarring agent is preferably not TGF-β3. The anti-scarring agent may be provided by intradermal N injection. Also provided are kits and methods of selecting an appropriate treatment regime for inhibiting scarring associated with the healing of a human wound.

The present invention relates to the provision of new methods for inhibiting scarring formed on healing of wounds. The invention also provides new uses of anti-scarring agents; new methods of selecting an appropriate treatment regime for inhibiting scarring associated with the healing of a wound; and kits for use in the inhibition of scarring associated with healing of a wound. The anti-scarring agent is preferably not TGF-β3.

The scarring response to healing of a wound is common throughout all adult mammals. The scarring response is conserved between the majority of tissue types and in each case leads to the same result, formation of fibrotic tissue termed a “scar”. A scar may be defined as “fibrous connective tissue that forms at the site of injury or disease in any tissue of the body”.

In the case of a scar that results from healing of a wound, the scar constitutes the structure produced as a result of the reparative response. This reparative process has arisen as the evolutionary solution to the biological imperative to prevent the death of a wounded animal. In order to overcome the risk of mortality due to infection or blood loss, the body reacts rapidly to repair the damaged area, rather than attempt to regenerate the damaged tissue. Since the damaged tissue is not regenerated to attain the same tissue architecture present before wounding, a scar may be identified by virtue of its abnormal morphology as compared to unwounded tissue.

Viewed macroscopically, scars may be depressed below the surface of the surrounding tissue, or elevated above the surface of their undamaged surroundings. Scars may be relatively darker coloured than normal tissue (hyperpigmentation) or may have a paler colour (hypopigmentation) compared to their surroundings. In the case of scars of the skin, either hyperpigmented or hypopigmented scars constitute a readily apparent cosmetic defect. It is also known that scars of the skin may be redder than unwounded skin, causing them to be noticeable and cosmetically unacceptable. It has been shown that the cosmetic appearance of a scar is one of the major factors contributing to the psychological impact of scars upon the sufferer, and that these effects can remain long after the wound that caused the scar has healed.

In addition to their psychological effects, scars may also have deleterious physical effects upon the sufferer. These effects typically arise as a result of the mechanical differences between scars and normal tissue. The abnormal structure and composition of scars mean that they are typically less flexible than their normal tissue counterpart. As a result scars may be responsible for impairment of normal function (such as in the case of scars covering joints which may restrict the possible range of movement) and may retard normal growth if present from an early age.

Despite the fact that the disadvantages associated with scarring are well known to those skilled in the art, there remains a requirement for new and improved methods of treatment that may be used to inhibit scarring associated with the healing of wounds.

It is an object of some aspects of the present invention to provide improved methods of inhibiting scarring formed on healing of wounds. It is an object of other aspects of the invention to provide new uses of anti-scarring agents. These new uses of anti-scarring agents may constitute alternative uses to those known from the prior art, but it may be preferred that they constitute improved uses compared to those already known. It is an object of certain aspects of the invention to provide; new methods of selecting an appropriate treatment regime for inhibiting scarring associated with the healing of a wound. It is an object of other aspects of the invention to provide kits for use in the inhibition of scarring associated with healing of a wound. These kits may be used in methods of treatment that provide increased inhibition of scarring compared to those known from the prior art.

In a first aspect of the invention there is provided a method of inhibiting scarring formed on healing of a wound, the method comprising treating a body site in which scarring is to be inhibited:
in a first incidence of treatment providing to each centimetre of wound margin, or each centimetre of a site at which a wound is to be formed a first therapeutically effective amount of an anti-scarring agent; and
in a second incidence of treatment, occurring after a wound is formed and between 8 and 48 hours after the first incidence of treatment, providing to said wound a therapeutically effective amount of said anti-scarring agent that is larger than the therapeutically effective amount of the anti-scarring agent provided in the first incidence of treatment.

In a second aspect, the invention provides a method of inhibiting scarring formed on healing of a wound, the method comprising treating a body site in which scarring is to be inhibited:
in a first incidence of treatment providing to each centimetre of a site where a wound is to be formed a first therapeutically effective amount of an anti-scarring agent; and
in a second incidence of treatment, occurring after a wound is formed and between 8 and 48 hours after the first incidence of treatment, providing to said wound a therapeutically effective amount of said anti-scarring agent that is larger than the therapeutically effective amount of the anti-scarring agent provided in the first incidence of treatment.

In a third aspect, the invention provides a method of inhibiting scarring formed on healing of a wound, the method comprising treating a body site in which scarring is to be inhibited:
in a first incidence of treatment providing to each centimetre of wound margin, or each centimetre of future wound margin, a first therapeutically effective amount of an anti-scarring agent; and
in a second incidence of treatment, occurring after a wound is formed and between 8 and 48 hours after the first incidence of treatment, providing to said wound a therapeutically effective amount of said anti-scarring agent that is larger than the therapeutically effective amount of the anti-scarring agent provided in the first incidence of treatment.

The present invention is based upon the inventors' finding that scarring that would otherwise be expected on healing of a wound can be surprisingly effectively inhibited by use of a treatment regime, comprising at least two incidences of treatment, in which the site where scarring is to be reduced is treated with larger therapeutically effective amounts of an anti-scarring agent in the second (and any subsequent) incidence of treatment than in the first. The first incidence of treatment may occur at a time around wounding or wound closure, and then each further incidence of treatment may occur between 8 and 48 hours after the preceding incidence. These treatment regimes, described for the first time in the present disclosure, give rise to scars that are much reduced compared to those obtainable using known methods of treatment.

Any of the aspects or embodiments of the present invention, including those describing medicaments, methods or kits, may preferably make use of an anti-scarring agent other than TGF-β3. It may be preferred that the aspects or embodiments of the invention utilise interleukin-10 (IL-10), or its fragments or derivatives, as the anti-scarring agent.

Without wishing to be bound by any hypothesis, the inventors believe that exposure of the cells at a wound, or a site where a wound will be formed, to the therapeutically effective amount of an anti-scarring agent provided in the first incidence of treatment is able to reduce the scarring response during the relatively early stages of wound healing. The anti-scarring agent provided in the second (and any further) incidence of treatment may serve to counteract the pro-scarring “cascade” of biological processes that otherwise arises at the wound site. Such cascades are typically self-amplifying, with various pro-fibrotic factors capable of bringing about their own induction or the induction of further factors that induce scarring. The use of a larger dose of the anti-scarring agent in the second incidence of treatment appears to counteract this amplification, and thus inhibit scarring more effectively than can be achieved using the methods of the prior art. The inventors' findings, set out in the Experimental Results section below, indicate that the use of anti-scarring agents, such as IL-10, in accordance with the invention may be able to reduce the number and/or proportion of inflammatory cells present in treated wounds. Since inflammatory cells are frequently implicated in the production of factors that may contribute to such “cascades” this may represent a possible mode of action by which the medicaments, methods and kits of the invention achieve their effects.

It is important to note that this mode of treatment has not been suggested before, possibly as result of teachings of the prior art that are discussed below. However, the inventors have found that this new approach has a surprisingly beneficial effect in inhibiting scarring, which is noticeably greater than the effects that may be achieved using other anti-scarring treatment regimes known to date.

The finding underlying the invention is highly surprising since not only are the anti-scarring results achieved particularly effective, but the prior art would have lead the skilled person to believe that this treatment regime using increasing doses of anti-scarring agents would not be of as much benefit as known regimes using smaller doses.

Previously it had been understood by those skilled in the art that the anti-scarring response elicited in response to anti-scarring agents took the form of a “bell shaped” dose response curve. This is exemplified by the response seen on administration of various single doses of the well known anti-scarring agent TGF-β3. Doses at the upper or lower ends of the dose response curve for TGF-β3 are not as effective as those positioned in the middle of the dose response. Based on these findings a preferred therapeutically effective amount of TGF-β3 to be provided as a single doses per centimetre of a site in which scarring was to be inhibited had been identified as approximately 200 ng. Single administrations of lower doses (of around 100 ng) or higher doses (such as 500 ng) did not give rise to such an effective reduction in scarring as did 200 ng. Investigations by the inventors, and by others working in this field, had determined that 200 ng doses of TGF-β3 are effective when administered prior to wounding, or to the wound margins after a wound is formed. This pattern of response, yielding a bell-shaped dose response curve, is observed with many other anti-scarring agents, and particularly other biological molecules (such as growth factors, growth factor neutralising agents, receptor ligands, or the like) that have anti-scarring activity.

Once studies into the anti-scarring effectiveness of agents such as TGF-β3 had identified the optimal dose to be used (for example 200 ng in the case of TGF-β3), further investigations considered whether any advantage was conferred by repeated administration of this dose to a site where scarring was to be reduced. These results showed that repeated administration of many anti-scarring agents, such as TGF-β3, to wounds generally did not provide any benefits in terms of the anti-scarring effect observed.

Given that the dose response curve had identified that increasing the dose of an anti-scarring agent (such as TGF-β3) administered to a wound (in a single dose treatment regime) would reduce the effectiveness of the treatment, any suggestion to use escalating doses of anti-scarring agents as part of a treatment regime would have been viewed as counterproductive. Based on the experiments that had been conducted (by the inventors and by other groups) it would have been anticipated that the use of multiple incidences of treatment would be no more effective than single treatments regimes, but only more complex and expensive. Furthermore, it would have been expected that a regime in which the amount of the anti-scarring agent provided to a wound was increased over time would actually reduce the effectiveness of the treatment since it would cause the amount of the anti-scarring agent provided to rise into the upper portions of the bell-curve, where increasing dosage actively decreased anti-scarring effectiveness.

In the light of the above, it can be seen that the skilled person had no motivation to consider treatments of the sort described herein, in which repeated incidences of treatment are utilised, and the amount of an anti-scarring agent provided to a body site at which scarring is to be inhibited increases between the first and second treatments. Thus, it will be appreciated that the findings set out in the present disclosure provide a surprising, but valuable, addition to the range of treatments that may be used to clinically inhibit the scarring of wounds.

Since the methods of treatment disclosed herein require at least two incidences of treatment, which take place between at least 8 to 48 hours apart from one another, they are not suitable for use in patients that would not be able to complete a second, or further incidence of treatment. This observation gives rise to a further aspect of the invention, in which there is provided a method of selecting an appropriate treatment regime for inhibiting scarring associated with the healing of a wound, the method comprising:
determining whether an individual in need of such inhibition of scarring will be able to complete a second incidence of treatment occurring between 8 and 48 hours after a first incidence of treatment; and
if the individual will be able to complete a second incidence of treatment occurring between 8 and 48 hours after a first incidence of treatment, selecting a treatment regime comprising a method of treatment in accordance with any of the first three aspects of the invention, or
if the individual will not be able to complete a second incidence of treatment occurring between 8 and 48 hours after a first incidence of treatment, selecting a treatment regime comprising:
in a single incidence of treatment providing to each centimetre of wound margin, or each centimetre of a site at which a wound is to be formed, in which scarring is to be inhibited, an amount of the anti-scarring agent that has been shown to be therapeutically effective when provided in a single incidence of treatment.

Therapeutically effective amounts of anti-scarring agents that may be used in a treatment regime comprising a single incidence of treatment may be identified by the skilled person with reference to the prior art. Merely by way of example, therapeutically effective amounts of a number of anti-scarring agents of particular interest are described elsewhere within the present specification.

In various aspects and embodiments of the invention, the present disclosure defines the amount of an anti-scarring agent to be provided to a body site with reference to the amount to be provided per centimetre of such a site (for example, per centimetre of a site to be wounded, or per centimetre of wound margin or of future wound margin). It will be appreciated that, while these passages define the amount of the anti-scarring agent to be provided to such sites, they do not limit the manner in which this amount is to be provided. In particular, these passages should not be taken as requiring the administration of the anti-scarring agent to each centimetre of a site to be treated (though this may be a preferred embodiment). The requisite amount of the anti-scarring agent may be provided by any number of administrations occurring at any site that allows the specified amount of the anti-scarring agent to be provided to the site at which scarring is to be inhibited.

In a further aspect of the invention there is provided an anti-scarring agent for use as a medicament in treating a wound or site where a wound is to be formed to inhibit scarring, wherein in a first incidence of treatment the medicament is provided such that a first therapeutically effective amount of the anti-scarring agent is provided to each centimetre of a wound margin or each centimetre of a site at which a wound is to be formed; and wherein in a subsequent incidence of treatment the medicament is provided such that a larger therapeutically effective amount of the anti-scarring agent is provided to each centimetre of a wound margin between 8 hours and 48 hours after the previous incidence of treatment.

In another aspect of the invention there is provided an anti-scarring agent for use as a medicament for treating a wound or site where a wound is to be formed to inhibit scarring, wherein in a first incidence of treatment the medicament is for provision such that a first therapeutically effective amount of the anti-scarring agent is provided to each centimetre of a wound margin or each centimetre of a site at which a wound is to be formed; and wherein in a subsequent incidence of treatment the medicament is provided such that a larger therapeutically effective amount of the anti-scarring agent is provided to each centimetre of a wound margin between 8 hours and 48 hours after the previous incidence of treatment.

In a still further aspect of the invention, there is provided interleukin-10 (IL-10), or a therapeutically effective fragment or derivative thereof, for use as a medicament for treating a wound or site where a wound is to be formed to inhibit scarring, wherein in a first incidence of treatment the medicament is provided such that a first therapeutically effective amount of the IL-10, or therapeutically effective fragment or derivative thereof, is provided to each centimetre of a wound margin or each centimetre of a site at which a wound is to be formed; and wherein in a subsequent incidence of treatment the medicament is provided such that a larger therapeutically effective amount of IL-10, or therapeutically effective fragment or derivative thereof, is provided to each centimetre of a wound margin between 8 hours and 48 hours after the previous incidence of treatment.

When it is desired to use IL-10, or a therapeutically effective fragment or derivative thereof, in accordance with this aspect of the invention, it may be preferred that the first therapeutically effective amount is between about ing and 1000 ng of IL-10 (or a therapeutically effective fragment or derivative thereof) per centimetre in human subjects. First incidences of treatment may suitably make use of a therapeutically effective amount of between about ing and 100 ng, between about 2 ng and 50 ng, or between about 5 ng and 25 ng per centimetre. Suitable therapeutically effective doses to be provided in a second incidence of treatment may be determined accordingly, with reference to the guidance provided elsewhere in the specification (for example, being up to 2, 3, 4, 5, 10, 20 or more times the size of the first therapeutically effective amount).

A medicament in accordance with this aspect of the invention may be a re-constitutable medicament, such as a lyophilised injectable composition.

The invention also provides use of an anti-scarring agent as a medicament in treating a wound or site where a wound is to be formed to inhibit scarring, wherein in a first incidence of treatment the medicament is provided such that a first therapeutically effective amount of the anti-scarring agent is provided to each centimetre of a wound margin or each centimetre of a site at which a wound is to be formed; and wherein in a subsequent incidence of treatment the medicament is provided such that a larger therapeutically effective amount of the anti-scarring agent is provided to each centimetre of a wound margin between 8 hours and 48 hours after the previous incidence of treatment.

When an anti-scarring agent is to be used in accordance with this aspect of the invention, it may be preferred that the medicament is an injectable medicament, and in particularly that the medicament is for intradermal injection.

Suitable medicaments formulated for use in any of the aspects of the invention may be such that the requisite amount of the anti-scarring agent is provided in a 100 μl volume of the medicament.

Furthermore, the inventors have found that the means for effecting the methods of the invention, including medicaments manufactured in accordance with the invention, may usefully be provided in the form of a kit for use in the inhibition of scarring associated with healing of a wound; the kit comprising at least first and second vials comprising an anti-scarring agent for administration to a wound, or a site where a wound is to be formed, at times between 8 and 48 hours apart from one another.

In a further aspect of the invention there is provided a kit for use in the inhibition of scarring associated with healing of a wound, the kit comprising:
a first amount of a composition containing an anti-scarring agent, this first amount being for administration to a wound, or a site where a wound is to be formed, in a first incidence of treatment;
a second amount of a composition containing the anti-scarring agent, this second amount being for administration to a wound in a second incidence of treatment;
instructions regarding administration of the first and second amounts of the composition at times between 8 and 48 hours apart from one another, and in a manner such that a larger therapeutically effective dose of the anti-scarring agent is administered to the wound in the second incidence of treatment than was administered in the first incidence of treatment.

A composition provided in such a kit may be provided in a form suitable for reconstitution prior to use (such as a lyophilised injectable composition).

It may be preferred that the first and second amounts of a composition respectively comprise different first and second compositions, wherein the second composition contains the anti-scarring agent at a greater concentration than does the first composition. In this case the instructions may indicate that a substantially similar volume of the first and second compositions should be administered to the site in the first and second incidences of treatment. Merely by way of example, the second composition may comprise the anti-scarring agent at a concentration that is approximately 10%, 20%, 30% or 40% greater than the concentration in the first composition; or even 50%, 60%, 70% 80% or 90% greater than the concentration in the first composition. The concentration of the anti-scarring agent in the second composition may be 100%, or more, greater than the concentration of the agent provided in the first composition.

Alternatively, the first and second compositions may contain the anti-scarring agent at substantially equal concentrations, and the instructions may indicate that the volume of the second composition administered in the second incidence of treatment should be larger than the volume of the first composition administered in the first incidence of treatment.

The inventors believe that the benefits that may be derived from the present invention may be applicable to wounds at sites throughout the body. However, it may be preferred that the wound, scarring associated with which is to be inhibited, is a skin wound. For illustrative purposes the embodiments of the invention will generally be described with reference to skin wounds, although they remain applicable to other tissues and organs. Merely by way of example, in another preferred embodiment the wound may be a wound of the circulatory system, particularly of a blood vessel (in which case the treatments may inhibit restenosis). Other wounds in which scarring may be inhibited in accordance with the present invention are considered elsewhere in the specification, and include those of the peripheral nervous system. The wound may be a result of surgery (such as elective surgery), and this constitutes a preferred embodiment of the invention.

The inventors believe that the methods, uses and kits disclosed in the present specification may be used in the inhibition of scarring in all animals, including human or non-human animals, such as domestic animals, sporting animals (such as horses) or agricultural animals. Wounds in which scarring is to be inhibited will preferably be those of a human subject.

The methods of the invention may optionally comprise a third or further incidence of treatment. Such further incidences of treatment may be continued as necessary until a clinician responsible for the care of the patient determines that a desired inhibition of scarring has been achieved. Each incidence of treatment should occur between 8 and 48 hours after the preceding incidence of treatment. Further guidance as to timing of third or further incidences of treatment may be taken from the disclosure herein relating to the relative timing of the first and second incidences.

The amount of a selected anti-scarring agent provided to the body site in a third incidence of treatment (and any further incidence of treatment) may be substantially the same as the amount provided in the second incidence of treatment (thus the dose provided effectively “plateaus” after the second incidence of treatment). Alternatively, the amount of the anti-scarring agent provided to the body site in the third (or subsequent) incidence of treatment may be larger than the amount provided in the preceding incidence of treatment (so that the amount of the anti-scarring agent provided escalates with each incidence of treatment).

There are a number of ways in which the methods of treatment of the invention may be put into practice, and these will be apparent to those of skill in the art. Certain preferred embodiments will now be described below by way of non-limiting examples. It will be appreciated that these examples are applicable to each of the first three aspects of the invention.

In one embodiment the first and second incidences of treatment (and other incidences as appropriate) may both make use of a composition comprising a given anti-scarring agent at substantially the same concentration. In this embodiment, the amount of the composition that is administered to the body site in the second incidence of treatment will be larger than the amount that is administered in the first incidence of treatment, and this difference provides the increase in dose between the different incidences.

It may be preferred that the first and second incidences of treatment (and, if appropriate any further incidences of treatment) make use of different compositions, wherein the composition used in the second incidence of treatment contains the anti-scarring agent at a greater concentration than does the composition used in the first incidence of treatment. In this case a substantially similar volume of the compositions containing the anti-scarring agent may be administered to the site in the first and second incidences of treatment (or even a smaller volume in the second incidence) since the increase in dose between the incidences occurs as a result of the increasing concentration of the anti-scarring agent in the compositions. Merely by way of example, the second (and further) incidences of treatment may make use of composition comprising the anti-scarring agent at a concentration that is approximately 10%, 20%, 30% or 40% greater than the concentration in the first composition; or even 50%, 60%, 70% 80% or 90% greater than the concentration in the first composition. The concentration of the anti-scarring agent in the second composition may even be 100%, or more, greater than the concentration of the agent provided in the first composition.

The therapeutically effective dose provided per centimetre of a body site (be it a site where a wound is to be formed, a wound margin, or a future wound margin) in the first incidence of treatment may be selected with reference to the particular anti-scarring agent that is being used. Suitable therapeutically effective amounts may be derived from the prior art, and certain illustrative examples in respect of various anti-scarring agents of particular interest are described elsewhere in the present specification.

The therapeutically effective dose of the anti-scarring agent provided per centimetre of body site in the second incidence of treatment may be approximately 10%, 20%, 30% or 40% greater than the therapeutically effective dose provided in the first incidence of treatment. The therapeutically effective amount of the anti-scarring agent provided in the second incidence of treatment may be 50%, 60%, 70% 80% or 90% greater than the therapeutically effective amount administered in the first incidence of treatment. The therapeutically effective amount of the anti-scarring agent provided in the second incidence of treatment may even be 100%, or more, greater than the therapeutically effective amount of the agent provided in the first incidence of treatment.

It will be appreciated that although the amount of an anti-scarring agent to be provided in each incidence of treatment is often referred to in the present disclosure on the basis of the amount to be provided per centimetre, the disclosure is not limited by this, and these references may be used to determine suitable doses that may be applied to a wound (or future wound) as measured by any suitable unit

It may be preferred that the first incidence of treatment occurs prior to wounding, in which case the anti-scarring agent may be provided to a site where a wound is to be formed. In the case that the anti-scarring agent is administered by local injection to the skin (such as intradermal injection) this may cause a bleb to be raised as a result of the introduction of a solution containing the anti-scarring agent into the skin. In one preferred embodiment the bleb may be raised in the site where the wound is to be formed, and indeed the wound may be formed by incising the bleb. In this case the amount of the anti-scarring agent to be provided in the first incidence of treatment may be determined with reference to the length of the site where the wound is to be formed.

Alternatively two blebs may be raised, on either side of the site where the wound is to be formed. These blebs may preferably be positioned within half a centimetre of where the margins of the wound will be formed. In this case the amount of the anti-scarring agent to be provided in the first incidence of treatment may be determined with reference to the length of the wound to be formed, measured in centimetres of future wound margin (defined below).

Preferably a bleb used to provide an anti-scarring agent to a site prior to wounding may cover substantially the full length of the site where the wound is to be formed. More preferably the bleb may extend beyond the length of the site where a wound is to be formed. Suitably such a bleb may extend around half a centimetre (or more) beyond each end of the wound to be formed.

Intradermal injections in accordance with these embodiments of the invention may be administered by means of a hypodermic needle inserted substantially parallel to the midline of the wound to be formed, or parallel to the margins of the wound to be formed. Injection sites may be spaced approximately one centimetre apart from one another along the length of the region to which the anti-scarring agent will be provided.

In the alternative, it may be preferred that the first incidence of treatment involves provision of the anti-scarring agent to an existing wound. The inventors believe that the biological mechanisms relevant to the anti-scarring activity are the same whether cells are exposed to the anti-scarring agent before or after wounding. In either case, the necessary biological activity may be achieved as long as the cells at the site where scarring is to be inhibited are exposed to a therapeutically effective amount of the anti-scarring agent either before or after wounding.

In embodiments of the invention in which the anti-scarring agent is to be provided to an existing wound, the requisite amount of the anti-scarring agent may be determined with reference to the length of the wound, measured in centimetres of wound margin (as discussed below). The anti-scarring agent should preferably be provided along the entire length of each wound margin, and may even be provided beyond the wounded area. In a preferred embodiment the anti-scarring agent may be provided along a length extending about half a centimetre (or more) beyond the ends of the margins of the wound.

Intradermal injection also represents a preferred route by which the anti-scarring agent may be administered to an existing wound. Intradermal injections administered in accordance with this embodiment should be administered to each margin of the wound. The site of injection may preferably be within half a centimetre of the edge of the wound. The injections may be administered by means of a hypodermic needle inserted substantially parallel to the edge of the wound. Injection sites may be spaced approximately one centimetre apart from one another along the length of the region to be treated.

The considerations set out in the preceding paragraphs in relation to provision of an anti-scarring agent to a wound in the first incident of treatment will also be applicable to its provision in second (or further) incidents. Since the second incidence of treatment takes place after wounding has occurred this will always involve provision of the anti-scarring agent to an existing wound. The wound may be open or closed, depending on the wound management strategy that is being applied.

When the first incidence of treatment involves provision of the anti-scarring agent to a site where a wound is to be formed it may be preferred that this provision occurs an hour or less before wounding is initiated, preferably half an hour or less before wounding is initiated, still more preferably a quarter of an hour or less before wounding is initiated, and most preferably ten minutes or less before wounding is initiated.

If the first incidence of treatment is to involve provision of the anti-scarring agent to an existing wound, the time at which this treatment is provided may be selected with reference to time elapsed after the wound has been formed. In this case, it may be preferred that a first incidence of treatment in accordance with the invention is initiated within two hours of wounding, preferably within one and a half hours of wounding, more preferably within an hour of wounding, still more preferably within half an hour of wounding, and most preferably within a quarter of an hour of wounding.

Alternatively or additionally, the timing of the first incidence of treatment may be selected with reference to the time elapsed after closure of the wound to be treated. In this case, it may be preferred that a first incidence of treatment in accordance with the invention is initiated within two hours of the closure of the wound being completed, preferably within one and a half hours of closure of the wound being completed, more preferably within an hour of closure of the wound being completed, still more preferably within half an hour of closure of the wound being completed, and most preferably within a quarter of an hour of closure of the wound being completed. In the case that a wound is not to be completely closed for clinical reasons (for example if it is necessary to maintain access to a site within the wound) closure of the wound may still be considered to have been completed once the wound is closed to the fullest extent that will be closed as part of the procedure undertaken.

It will be appreciated that selection of the timing of the first incidence of treatment with reference to the time elapsed after closure of the wound may be of particular relevance in the case of protracted surgical procedures, where a wound must be kept open for a prolonged time in order to allow access to a site where surgery is being performed.

The time elapsing between incidences of treatment will be between 8 and 48 hours. More preferably the time elapsing should be at least 10 hours, even more preferably at least 12 hours, yet more preferably at least 14 hours, still more preferably at least 16 hours, yet more preferably still at least 18 hours, more preferably still at least 20 hours, ever more preferably at least 22 hours, and most preferably is approximately 24 hours.

The time elapsing between incidences of treatment may be up to 48 hours, but will preferably be up to approximately 44 hours, more preferably up to approximately 40 hours, even more preferably up to approximately 36 hours, yet more preferably up to approximately 32 hours, still more preferably up to approximately 28 hours, and most preferably is approximately 24 hours.

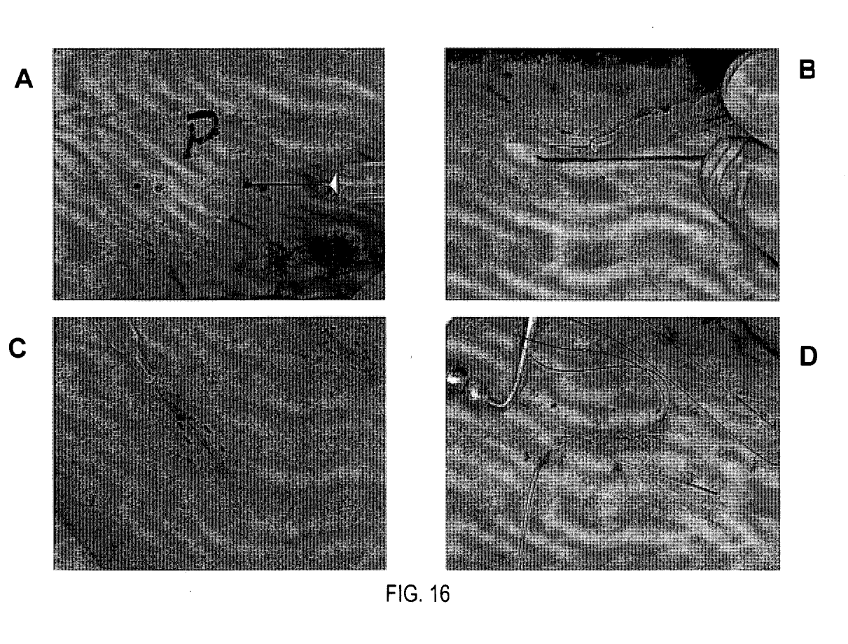

In practicing the methods of the invention, the cells of the area in which scarring is to be inhibited should be “bathed” in a pharmaceutically acceptable solution comprising a therapeutically effective amount of the anti-scarring agent. This will create a local environment in which the cells are exposed to sufficient of the anti-scarring agent to prevent scarring. Cells that would otherwise be involved in scar formation will receive the therapeutically effective amount of the selected anti-scarring agent whether the agent is administered by injection at the margins of a wound (or along the margins of a future wound—technique shown in panel B of FIG. 16), or by injection directly into the site at which the wound is to be formed (for example, by raising a bleb covering the site to be wounded—technique shown in panel A of FIG. 16). Either of these routes of administration are able to establish an anti-scarring concentration of the selected agent in the area surrounding the cells.

When the first incidence of treatment utilises injection directly into the site to be wounded, the requisite amount of the anti-scarring agent may be established around the cells by administration of a single injection (or series of “single” injections) administered along the line of the future wound and which cover the area to be wounded (technique illustrated in panel A of FIG. 16). When the first incidence of treatment utilises “paired” injections to each margin of a wound (or “paired” injections down each future margin of a wound—technique illustrated in panel B of FIG. 16) it will be appreciate that the total amount of the anti-scarring agent to be administered will be larger than that provided via the single injection route (described above), since injections on each margin are required in order to treat the same area.

It is preferred that the anti-scarring agent be provided to the requisite body site in the methods of the invention by means of an administration of a suitable pharmaceutical composition. Preferred compositions may be those suitable for injection, and in particular for intradermal injection. Many formulations of compositions that may be used for the administration of anti-scarring agents by intradermal injection will be known to those skilled in the art, and these may be selected with reference to the particular anti-scarring agent of interest.

Various terms used in the present disclosure will now be described further for the avoidance of doubt. It will be appreciated that, for the sake of brevity, some of these terms may be described with reference to only certain aspects of the invention. However, except for where the context requires otherwise, the following descriptions of these terms will be applicable to all aspects of the invention.

Centimetre of a Site Where a Wound is to be Formed

For ease of reference, the length of a site where a wound is to be formed may be measured in centimetres in order to determine the amount of the anti-scarring agent that will need to be provided in order to reduce scarring in accordance with the invention. It may be preferred that the length to be treated be calculated to extend beyond the intended length of the wound to be formed, in order to ensure that a therapeutically effective amount of the anti-scarring agent is provided to the ends of the wound. Accordingly, it may be preferred that the calculated length of a site where a wound is to be formed (and hence the length of the site to be treated) extend by a distance of about half a centimetre (or more) beyond each end of the intended wound.

Centimetre of Future Wound Margin

For the purposes of the present disclosure the length of a site where a wound is to be formed, as measured in number of centimetres of future wound margin, should be calculated as the sum of the lengths of each margin of the wound to be formed (in centimetres). It may be preferred that the length to be treated be calculated to extend beyond the ends of the margins of the wound to be formed, and this may help to ensure that a therapeutically effective amount of the anti-scarring agent is provided to the ends of the wound. Accordingly, it may be preferred that the calculated length of a future wound margin (and hence the length of the site to be treated) extend by a distance of about half a centimetre (or more) at each end of the wound to be formed.

Anti-Scarring Agents

The inventors believe that the various aspects and embodiments of the invention may be of benefit in connection with almost any anti-scarring agents identified in the prior art. “Biological” anti-scarring agents (i.e. naturally occurring anti-scarring agents or those based on such agents, such as growth factors, growth factor receptors, or the like) may be particularly suited to employment in accordance with the present invention.

Anti-scarring growth factors represent preferred anti-scarring agents for use in accordance with the present invention. The ability of these agents to inhibit scarring in this manner is particularly surprising since anti-scarring growth factors frequently have “bell-shaped” dose response curves (of the sort described elsewhere in the specification) suggesting that increasing doses of the agent would have little anti-scarring activity.

The selected anti-scarring agent to be used in accordance with the present invention (whether in the methods, uses or kits of the invention) is preferably not TGF-β3.

Merely by way of non-limiting example, the inventors believe that the methods, uses and kits described in the present disclosure may be advantageously used with at least some of the anti-scarring agents considered in the following paragraphs.

Suitable anti-scarring agents for use in accordance with the present invention, whether in the medicaments, methods, uses or kits, may be selected from the group consisting of: agents capable of neutralising pro-fibrotic growth factors independently selected from the group consisting of PDGF, TGF-β1 and TGF-β2; mannose 6 phosphate, and compounds related to this agent; soluble TGF-β3 receptors, or fragments thereof, such as soluble betaglycan; interleukin-10 (IL-10), its fragments and derivatives; inhibitors of interferon-gamma; agents that are able to influence the sex hormone system in such a manner as to inhibit scarring; agents capable of severing extracellular activin; agents capable of neutralising oestrogenic activity and/or promoting progesterone activity: the latency associated peptide (LAP) of TGF-β; inhibitors of convertase enzymes, such as furin; antagonists of CXCL13 or CXCR5 activity; WNT5A, or therapeutically effective fragments or derivatives thereof; antagonists of LXR; antagonists of FXR; WNT3A, or a therapeutically effective fragment or derivative thereof; sFRP3, or a therapeutically effective fragment or derivative thereof; and agonists of a member of the nuclear hormone receptor NR4A subgroup.

Agents Capable of Neutralising PDGF, TGF-β1 and/or TGF-β2

Agents capable of neutralising pro-fibrotic growth factors independently selected from the group consisting of PDGF, TGF-β1 and TGF-β2 represent suitable anti-scarring agents that may be employed in the manner described in the present disclosure. Merely by way of example, such agents may include neutralising antibodies having the required specificity, agents capable of interfering with the binding of these growth factors to their receptors, or agents capable of preventing expression of these growth factors (including antisense oligonucleotides, SiRNA, or the like). Further details of the use of agents capable of neutralising PDGF, TGF-β1 and/or TGF-β2 in the inhibition of scarring may be found in the inventors' earlier patent U.S. Pat. No. 5,662,904 (the disclosure of which is incorporated herein by reference insofar as it relates to the identification of effective anti-scarring agents, or therapeutically effective amounts of such agents). Merely by way of example the inventors believe that a therapeutically effective amount of such an agent to be administered to a centimetre of a body site where scarring is to be inhibited may comprise sufficient of the agent to neutralise an amount of between 1 μg and 1 μg of the pro-fibrotic growth factor(s).

Mannose 6 Phosphate and Related Compounds

The inventors believe that mannose 6 phosphate, and compounds related to this agent, may represent suitable anti-scarring agents that may be employed in the manner described in the present disclosure.

The compounds disclosed in the inventors' U.S. Pat. No. 6,140,307, U.S. Pat. No. 6,566,339 and U.S. Pat. No. 6,900,181 represent particular compounds related to mannose 6 phosphate that may be preferred anti-scarring agents in accordance with the present invention. The disclosure of these documents is incorporated herein by reference insofar as it relates to the identification of effective anti-scarring agents, or therapeutically effective amounts of such agents. For guidance, the inventors believe that a therapeutically effective amount of the compounds disclosed in these patents may be provided by administration of approximately 100 μl of a 10 mM, 20 mM, or preferably a 40 mM solution per centimetre of a site at which scarring is to be inhibited.

Soluble TGF-β Receptors

Soluble TGF-β3 receptors, or fragments thereof, such as soluble betaglycan may represent preferred anti-scarring agents that may be employed in accordance with all aspects of the present invention. Further details of the use of soluble TGF-β receptors as anti-scarring agents may be found in the inventors' earlier patents, such as U.S. Pat. No. 6,060,460 (the disclosure of which is incorporated herein by reference insofar as it relates to the identification of effective anti-scarring agents, or therapeutically effective amounts of such agents). Merely by way of example, the inventors believe that a therapeutically effective amount of soluble betaglycan for use as an anti-scarring agent may comprise approximately 1 μg and 10 μg of soluble betaglycan per centimetre of body site at which scarring is to be inhibited.

Interleukin-10 and Related Peptides

The inventors believe that interleukin-10 (IL-10), its fragments and derivatives constitute preferred anti-scarring agents that may be employed in accordance with the present invention in its various aspects and embodiments. It is preferred that the IL-10, or fragment or derivative thereof, is human IL-10, or is derived therefrom. The amino acid sequence of human IL-10 is shown in Sequence ID No. 3, and the sequence of DNA encoding human IL-10 is shown in Sequence ID No. 4. Fragments and derivatives of IL-10 that may be used in accordance with the various aspects or embodiments of the present invention include any that are therapeutically effective (which, for the purposes of the present disclosure, includes any fragments or derivatives of IL-10 capable of inhibiting scarring). For example, a partially modified form of IL-10, that differs from IL-10 by the addition, substitution or deletion of at least one amino acid, and that has at least 95% homology with IL-10, may be used as a preferred anti-scarring agent. Suitable fragments or derivatives of IL-10 may preferably retain the anti-inflammatory healing functionality of IL-10. Fragments and derivatives of IL-10 that may constitute anti-scarring agents of particular interest are disclosed in the inventors' earlier patents (e.g. U.S. Pat. No. 6,387,364, U.S. Pat. No. 7,052,684 or WO2006/075138). The disclosure of these patents is incorporated herein by reference insofar as it relates to the identification of effective anti-scarring agents, or therapeutically effective amounts of such agents.

Merely by way of example, the inventors believe that a therapeutically effective amount of the compounds disclosed in these patents may be provided by administration of approximately 100 μl of a 1 μM to 10 μM solution per centimetre of a site at which scarring is to be inhibited.

In animal models of scarring, the inventors have identified that a suitable first therapeutically effective amount of IL-10 (or a fragment or derivative thereof) may be between approximately 100 ng and 5000 ng, and the second therapeutically effective amount may be between approximately 200 ng and 10000 ng (bearing in mind that the second therapeutically effective amount must always be larger than the first therapeutically effective amount). For instance, the first therapeutically effective amount may be between about 250 and 2500 ng, while the second therapeutically effective amount may be between about 750 and 7500 ng.

The inventors have found that in humans even lower doses of IL-10, or therapeutically effective fragments or derivatives thereof, may be therapeutically effective. Merely by way of example, it may be wished to use a dose of between 1 ng and 1000 ng of IL-10 (or a therapeutically effective fragment or derivative thereof) per centimetre in a first incidence of treatment in humans, with larger therapeutically effective doses to be provided in a second incidence of treatment determined accordingly. First incidences of treatment may suitably make use of a therapeutically effective amount of between about ing and 100 ng, between about 2 ng and 50 ng, or between about 5 ng and 25 ng.

Inhibitors of Interferon-Gamma

Inhibitors of interferon-gamma have previously been shown by the inventors to represent agents that may be used to inhibit scarring. The inventors believe that such inhibitors (particularly neutralising antibodies, antisense oligonucleotides, SiRNA, or the like) may represent anti-scarring agents that are suitable for employment in the manners considered in the present disclosure. Details of such agents are considered in the inventors' own previous patents, such as U.S. Pat. No. 7,220,413 (the disclosure of which is incorporated herein by reference insofar as it relates to the identification of effective anti-scarring agents, or therapeutically effective amounts of such agents). Merely by way of guidance, the inventors believe that a therapeutically effective amount of such an inhibitor (suitable for provision to a centimetre of a body site in order to inhibit scarring) may be an amount capable of inhibiting the activity of between 300 and 30000 IU of interferon-gamma.

Activin and Inhibin

The TGF-β3 superfamily members activin and inhibin represent an anti-scarring agents, and the inventors believe that these proteins (or their therapeutically effective fragments or derivatives) may represent anti-scarring agents that may be usefully employed in the various aspects of the invention disclosed herein. Further details regarding the anti-scarring use of activin or inhibin may be found in the inventors' earlier patents, such as EP 0855916 (the disclosure of which is incorporated herein by reference insofar as it relates to the identification of effective anti-scarring agents, or therapeutically effective amounts of such agents).

Agents that Influence the Sex Hormone System

The inventors' earlier patents and applications (such as WO 98/03180; the disclosure of which is incorporated herein by reference insofar as it relates to the identification of effective anti-scarring agents, or therapeutically effective amounts of such agents) disclose various agents that are able to influence the sex hormone system in such a manner as to inhibit scarring. The inventors believe that any of these agents may be suitable for employment in the manner described in the present specification, and that agents capable of neutralising oestrogenic activity and/or promoting progesterone activity are particularly suitable anti-scarring agents for use in the various aspects and embodiments of the invention.

Agents Capable of Severing Extracellular Activin

Various actin-severing proteins have been shown to be able to function as anti-scarring agent (particularly when functioning extracellularly), as described in the inventors' earlier patents, such as EP 0941108 (the disclosure of which is incorporated herein by reference insofar as it relates to the identification of effective anti-scarring agents, or therapeutically effective amounts of such agents). Gelsolin represents a preferred example of an actin-severing protein that may be used as such an anti-scarring agent. The inventors believe that treatment with escalating doses of gelsolin (or other actin-severing proteins) in the manner contemplated in the present disclosure may provide notable advantages in terms of the anti-scarring activity that can be achieved using this agent. Merely by way of example, the inventors believe that a therapeutically effective amount of the compounds disclosed in these patents may be provided by administration of approximately 100 μl of a 50 nM to 1000 nM solution per centimetre of a site at which scarring is to be inhibited.

LAP

The latency associated peptide (LAP) of TGF-β3 may represent a suitable anti-scarring agent that may be advantageously employed in the methods, uses and kits of the present invention. Details of the use of LAP as an anti-scarring agent are disclosed in the inventors' own patent applications, such as U.S. Pat. No. 6,319,907 (the disclosure of which is incorporated herein by reference insofar as it relates to the identification of effective anti-scarring agents, or therapeutically effective amounts of such agents). The inventors believe that a therapeutically effective amount of LAP able to inhibit scarring in a centimetre of a body site to which it is provided may be in the region of ing to 10 mg.

Inhibitors of Convertase Enzymes

The inventors have previously described (for example in WO 2004009113) how inhibitors of convertase enzymes (such as furin) may be used as agents having anti-scarring activity. The particular agents (such as isdecanoyl-RVKR-cmk and hexa-arginine) and criteria to be used in selection of convertase inhibitors disclosed in that application are believed to represent anti-scarring agents that may provide unexpectedly increased anti-scarring activity when employed in the manner described in the present disclosure. The contents of this earlier application, insofar as they relate to suitable agents or the selection of suitable agents are to be taken as incorporated by reference herein. Purely for guidance, the inventors believe that a therapeutically effective amount of a convertase inhibitor such as decanoyl-RVKR-cmk may be provided by administration of approximately 100 μl of a 0.1 μM and 10 mM solution per centimetre of a site at which scarring is to be inhibited.

Antagonists of CXCL13 or CXCR5 Activity

The inventors have found that agents capable of antagonising CXCL13 or CXCR5 activity are capable of inhibiting scarring, as described in WO 2007/122402. This earlier patent application provides details of doses and particularly preferred antagonists that may be used to reduce scarring. Accordingly, antagonists of CXCL13 or CXCR5 activity (and particularly those disclosed in WO 2007/122402) are considered to be anti-scarring agents that may be employed in accordance with the present invention. The disclosure of WO 2007/122402 (in particular as it relates to preferred anti-scarring agents and therapeutically effective amounts of such agents) is incorporated herein by reference.

WNT5A

The inventors believe that WNT5A (or therapeutically effective fragments or derivatives thereof) constitutes an anti-scarring agent that may gain additional anti-scarring activity through use in accordance with the various aspects or embodiments of the present invention (whether methods, uses or kits).

WNT5A may preferably be provided at less than 2000 ng per centimetre of a body site where it is wished to inhibit scarring, in order to provide a therapeutically effective amount of the selected anti-scarring agent.

The disclosure of the inventors' own earlier patent application (filed as PCT/GB2007/002445, and incorporated herein by reference) may be used to provide guidance as to preferred anti-scarring agents that may be used in accordance with this embodiment, and to therapeutically effective amounts of such agents.

Antagonists of LXR

The inventors have identified that antagonists of LXR may be used to inhibit scarring. This is more fully described in the inventors' earlier patent application GB 0625965.9. The inventors believe that antagonists of LXR may be employed in accordance with the present invention, and the contents of their earlier patent application, insofar as they relate to the selection of suitable anti-scarring agents and therapeutically effective amounts of such agents, are herein incorporated by reference.

Suitable anti-scarring agents in accordance with this embodiment of the invention include fibrate ester; geranylgeranyl pyrophosphate, Riccardin F, an auto-oxidised cholesterol sulphate, Wy-14643, 7-ketocholesterol-3-sulfate, and 5α,6α-epoxycholesterol-3-sulfate. A therapeutically effective amount of an antagonist of LXR may be between about 13 pmoles and about 2 nmoles of the antagonist per centimetre of a body site at which it is desired to inhibit scarring (preferably provided over a 24 hour period)

Antagonists of FXR Activity

The inventors have identified that antagonists of FXR may be used to inhibit scarring. This is more fully described in the inventors' earlier patent application GB 0625966.7. The inventors believe that antagonists of FXR may be employed in accordance with the present invention, and the contents of their earlier patent application, insofar as they relate to the selection of suitable anti-scarring agents and therapeutically effective amounts of such agents, are herein incorporated by reference.

Preferred anti-scarring agents in accordance with this embodiment of the invention include those selected from the group consisting of guggulsterone (Z); guggulsterone (E); a scalarane; 80-574; and a 5α-bile alcohol. Such anti-scarrring agents may be provided in a therapeutically effective amount of up to 32 μM of the antagonist per centimetre of a body site at which it is desired to inhibit scarring over a 24 hour period

WNT3A

The inventors have found that WNT3A (or a therapeutically effective fragment or derivative thereof) may be used to inhibit scarring, and believe that such agents may represent anti-scarring agents that may be employed in accordance with the present invention. This is more fully described in the inventors' earlier patent application GB 0702930.9, and the contents of their earlier patent application, insofar as they relate to the selection of suitable anti-scarring agents and therapeutically effective amounts of such agents, are herein incorporated by reference. Merely by way of example, such agents may be provided in an amount of approximately 1 ng per centimetre of a site where scarring is to be inhibited, in order to provide a therapeutically effective amount.

sFRP3

The inventors have found that sFRP3 (or a therapeutically effective fragment or derivative thereof) may be used to inhibit scarring, and believe that such agents may represent anti-scarring agents that may be employed in accordance with the present invention. This is more fully described in the inventors' earlier patent application GB 0707348.9, and the contents of their earlier patent application, insofar as they relate to the selection of suitable anti-scarring agents and therapeutically effective amounts of such agents, are herein incorporated by reference. Merely by way of example, such agents may be provided in an amount of between about 2.6 fmol and 40 pmol per centimetre of a site where scarring is to be inhibited, in order to provide a therapeutically effective amount.

NR4A Agonists

The inventors have found that agonists of a member of the nuclear hormone receptor NR4A subgroup may be used to inhibit scarring, and believe that such agents may represent anti-scarring agents that may be employed in accordance with the present invention. This is more fully described in the inventors' earlier patent application GB 0714934.7, and the contents of their earlier patent application, insofar as they relate to the selection of suitable anti-scarring agents and therapeutically effective amounts of such agents, are herein incorporated by reference. Merely by way of example, 6-mercaptopurie may represent a preferred anti-scarring agent in accordance with this embodiment, and may be provided in an amount of between about 0.59 pmol and 8.85 nmol per centimetre of a site where scarring is to be inhibited, in order to provide a therapeutically effective amount.

Centimetre of Wound Margin

For the purposes of the present disclosure, the length of a wound, as measured in number of centimetres of wound margin, should be calculated as the sum of the lengths of each margin of the wound (in centimetres). It may be preferred that the length of the site to be treated be calculated to extend beyond the ends of the margins of the wound. This may help to ensure that a therapeutically effective amount of the anti-scarring agent is provided to the ends of the wound. Accordingly, it may be preferred that the calculated length of a wound margin to be treated in accordance with the invention extend by a distance of about half a centimetre (or more) beyond each end of the wound.

“Therapeutically Effective Amounts”

A therapeutically effective amount of an anti-scarring agent for the purposes of the present disclosure is any amount of an anti-scarring agent that is able to prevent, reduce or inhibit scarring associated with healing of a wound when used in accordance with the present invention. It will be appreciated that amounts of anti-scarring agents that are not therapeutically effective when considered in, for example, dose response experiments using single administrations of the agent may still be therapeutically effective in a model of scarring using two incidences of treatment, as described in the present specification.

Guidance as to therapeutically effective amounts of particular anti-scarring agents that it may be wished to employ in accordance with the present invention (for example in the methods, uses or kits of the present invention) may be found in the prior art, by experimentation, or with reference to guidance provided elsewhere in the current specification. It will be appreciated that amounts of such anti-scarring agents that have previously been shown to have therapeutic activity may be used as therapeutically effective amounts suitable for provision to a body site where scarring is to be inhibited in a first incidence of treatment, or as the starting point for experiments intended to define suitable therapeutically effective amounts that may be provided in the first incidence of treatment.

Prevention/Inhibition/Reduction/Minimisation of Scarring

The inhibition of scarring within the context of the present invention should be understood to encompass any degree of prevention, reduction, minimisation or inhibition in scarring achieved on healing of a wound treated in accordance with a method of the invention (or a kit or medicament of the invention) as compared to the level of scarring occurring on healing of a control-treated or untreated wound. For the sake of brevity, the present specification will primarily refer to “inhibition” of scarring utilising anti-scarring agents, however, such references should be taken, except where the context requires otherwise, to also encompass the prevention, reduction or minimisation of scarring using these anti-scarring agents.

Pharmaceutically Acceptable

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the US Federal or a state government or listed in the U.S. Pharmacopoeia or other generally recognized pharmacopeias for use in animals, and more particularly in humans.

Pharmaceutical Compositions and Administration

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. The invention therefore includes within its scope pharmaceutical compositions comprising a product of the present invention that is adapted for use in human or veterinary medicine.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Wounds

The inventors believe that methods of treatment using in accordance with the present invention may be used to beneficially inhibit scarring in all types of wounds.

Examples of specific wounds in which scarring may be inhibited using the medicaments and methods of the invention include, but are not limited to, those independently selected from the group consisting of wounds of the skin; wounds of the eye (including the inhibition of scarring resulting from eye surgery such as LASIK surgery, LASEK surgery, PRK surgery, glaucoma filtration surgery, cataract surgery, or surgery in which the lens capsule may be subject to scarring) such as those giving rise to corneal cicatrisation; wounds subject to capsular contraction (which is common surrounding breast implants); wounds of blood vessels; wounds of the central and peripheral nervous system (where prevention, reduction or inhibition of scarring may enhance neuronal reconnection and/or neuronal function); wounds of tendons, ligaments or muscle; wounds of the oral cavity, including the lips and palate (for example, to inhibit scarring resulting from treatment of cleft lip or palate); wounds of the internal organs such as the liver, heart, brain, digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision). It is particularly preferred that the medicaments and methods of the invention be used to prevent, reduce, inhibit or minimise scarring associated with wounds of the skin.

Particular anti-scarring agents that may be used in accordance with the present invention may be of increased utility in certain types of wounds. Guidance as to particular anti-scarring agents that may be of benefit in treating a particular type of wound of interest may be taken from reported activities of the anti-scarring agents in the prior art.

Assessment of Scarring

The extent of scarring, and so any inhibition of scarring achieved, may be assessed by macroscopic clinical assessment of scars. This may be achieved by the direct assessment of scars upon a subject; or by the assessment of photographic images of scars; or of silicone moulds taken from scars, or positive plaster casts made from such moulds. For the purposes of the present disclosure a “treated scar” should be taken to comprise a scar produced on healing of a wound treated in accordance with the present invention.

Suitable assessment of scarring, and hence inhibition of scarring indicative that a putative agent is suitable for use as anti-scarring agent as described herein, may be undertaken in human subjects or in appropriate animal models. The use of animal models for investigation of the activity of anti-scarring agents subsequently intended for use in human subjects is well documented and scientifically accepted.

Macroscopic characteristics of a scar which may be considered when assessing scarring include:
i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Scars may often be redder than the surrounding skin. In this case inhibition of scarring may be demonstrated when the redness of a treated scar fades earlier, or more completely, or to resemble more closely the appearance of the surrounding skin, compared to an untreated scar. Colour can readily be measured, for example by use of a spectrophotometer.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on the patient (e.g. by means of profilometry), or indirectly, (e.g. by profilometry of moulds taken from a scar).
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can also be measured either directly on the patient (e.g. by means of profilometry), or indirectly (e.g. by profilometry of moulds taken from a scar).
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two of the parameters, even more preferably at least three of the parameters, and most preferably at least four of these parameters (for example, all four of the parameters set out above).

The height, length, width, surface area, depressed and raised volume, roughness/smoothness of scars can be measured directly upon the subject, for example by using an optical 3D measurement device. Scar measurements can be made either directly on the subject, or on moulds or casts representative of the scar (which may be formed by making a silicone mould replica impression of the scar and subsequently creating a plaster cast from the silicone moulds). All of these methods can be analysed using an optical 3D measurement device, or by image analysis of photographs of the scar. 3D optical measurements have a resolution in the micrometer range along all axes which guarantees a precise determination of all skin and scar parameters. The skilled person will also be aware of further non-invasive methods and devices that can be used to investigate suitable parameters, including calipers for manual measurements, ultrasound, 3D photography (for example using hardware and/or software available from Canfield Scientific, Inc.) and high resolution Magnetic Resonance Imaging.

Inhibition of scarring may be demonstrated by a reduction in the height, length, width, surface area, depressed or raised volume, roughness or smoothness or any combination thereof, of a treated scar as compared to an untreated scar.

One preferred method for the macroscopic assessment of scars is holistic assessment. This may be accomplished by means of assessment of macroscopic photographs by an expert panel or a lay panel, or clinically by means of a macroscopic assessment by a clinician or by patients themselves. Assessments may be captured by means of a VAS (visual analogue scale) or a categorical scale. Examples of suitable parameters for the assessment of scarring (and thereby of any reduction of scarring attained) are described below. Further examples of suitable parameters, and means by which assessment of such parameters may be captured, are described by Duncan et al. (2006), Beausang et al. (1998) and van Zuijlen et al. (2002).

Assessment Using Visual Analogue Scale (VAS) Scar Scores.

Assessments of scars may be captured using a scarring-based VAS. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar. Use of a VAS in this manner allows for easy capture and quantification of assessment of scarring. VAS scoring may be used for the macroscopic and/or microscopic assessment of scarring.

Merely by way of example, a suitable macroscopic assessment of scarring may be carried out using a VAS consisting of a 0-10 cm line representing a scale, from left to right, of 0 (corresponding to normal skin) to 10 (indicative of a bad scar). A mark may be made by an assessor on the 10 cm line based on an overall assessment of the scar. This may take into account parameters such as the height, width, contour and colour of the scar. The best scars (typically of small width, and having colour, height and contour like normal skin) may be scored towards the “normal skin” end of the scale (the left hand side of the VAS line) and bad scars (typically large width, raised profile and with uneven contours and whiter colour) may be scored towards the “bad scar” end of the scale (the right hand side of the VAS line). The marks may then be measured from the left hand side to provide the final value for the scar assessment in centimetres (to 1 decimal place).

An alternative assessment of scarring (whether macroscopic assessment or microscopic assessment), involving the comparison of two scars or two scar segments (such as one treated segment and another segment untreated, or control treated) to determine which one has a preferred appearance, may be carried out using a VAS comprising two 100 mm VAS lines intersected by a vertical line. In a VAS of this sort, the two VAS lines correspond to the two scars being compared, while the vertical line represents zero (indicating that there is no perceptible difference between the scars compared). The extremes of 100% (100 mm at the end of either VAS line) indicate one of the scars has become imperceptible in comparison to the surrounding skin.

A particularly preferred method of assessing the macroscopic appearance of scars in this manner is referred to as The Global Scar Comparison Scale (GSCS). This scale has been positively received by the European Medicines Agency (EMEA) and accepted as a preferred scale by which scars may be assessed and clinically relevant endpoints associated with the inhibition of scarring determined. In particular, it may be preferred to use a version of the GSCS based on clinical panel assessment, this being viewed by the EMEA as particularly relevant.

When comparing a pair of scars using a VAS of this sort, such as the GSCS, an assessor must first decide which of the scars has the preferred appearance, or if there is no perceptible difference between the two. If there is no perceptible difference this is recorded by placing a mark at the zero vertical line. If there is a perceptible difference, the assessor uses the worse of the two scars as an anchor to determine the level of improvement found in the preferred scar, and then marks the score on the relevant section of the scale. The point marked represents the percentage improvement over the anchor scar.

The inventors have found that use of VAS measures of this sort in assessing the macroscopic or microscopic appearance of scars offers a number of advantages. Since these VAS are intuitive in nature they, 1) reduce the need for extensive training using reference images of different scar severities in different skin types, making this tool relatively simple to deploy in a large phase 3 trial; 2) reduce variability of the data: one assessment of each scar pair is performed as opposed to two independent assessments of drug and placebo scars; 3) incorporate the well-established principles of VAS (i.e., a continuous distribution of data) and the benefits of ranking in the same scale; and 4) allow easier communication of drug effect (percentage improvement) to clinicians and patients.

The present invention will now be further described with reference to the following Experimental Results section, and accompanying drawings. The Experimental Results section illustrates the efficacy of the present invention with reference to the well-known anti-scarring agent TGF-133, though it may be preferred that the invention (in any of its aspects or embodiments) make use of an anti-scarring agent other than TGF-β3.

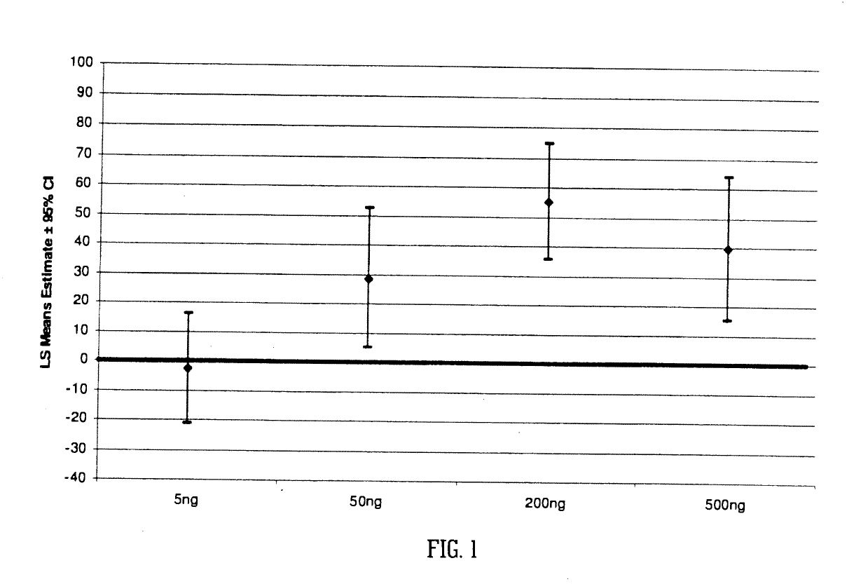

FIG. 1 compares the anti-scarring activity of different doses of TGF-β3 provided to human wounds in a single incidence of treatment.

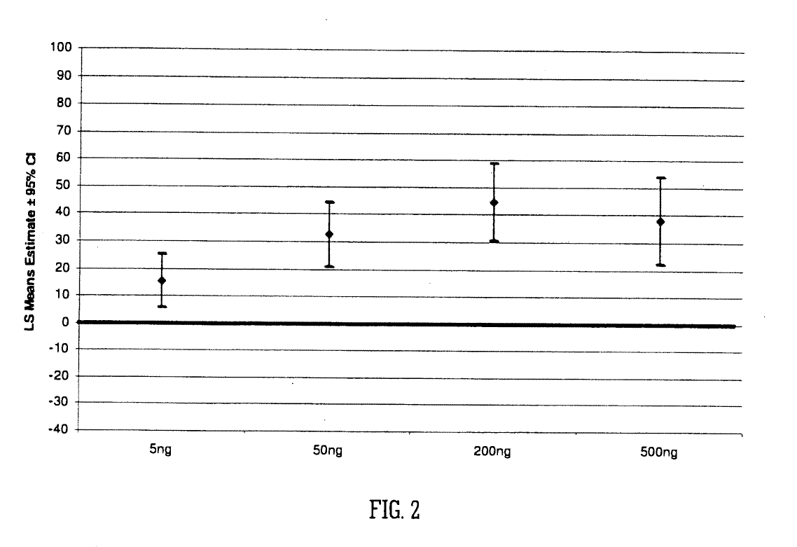

FIG. 2 compares the anti-scarring activity of different doses of TGF-β3 provided to human wounds in two incidences of treatment administered within approximately one hour of one another.

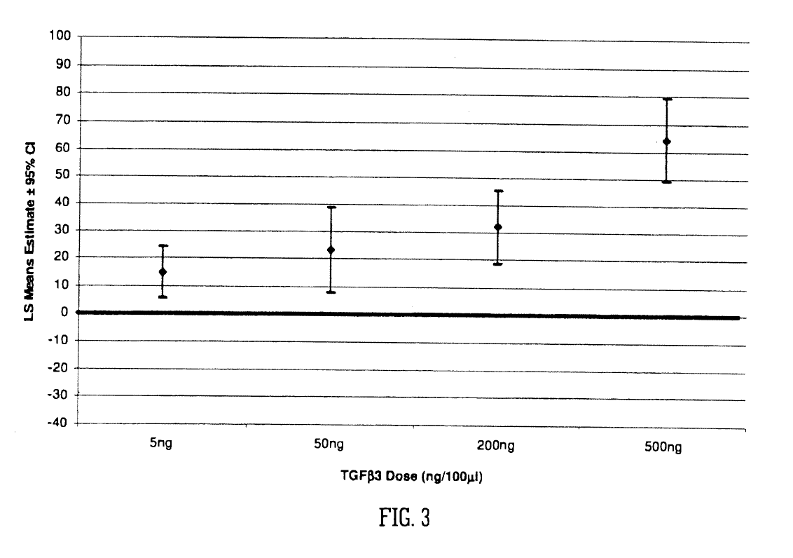

FIG. 3 compares the anti-scarring activity of different doses of TGF-β3 provided to human wounds in two incidences of treatment administered approximately 24 hours apart from one another.

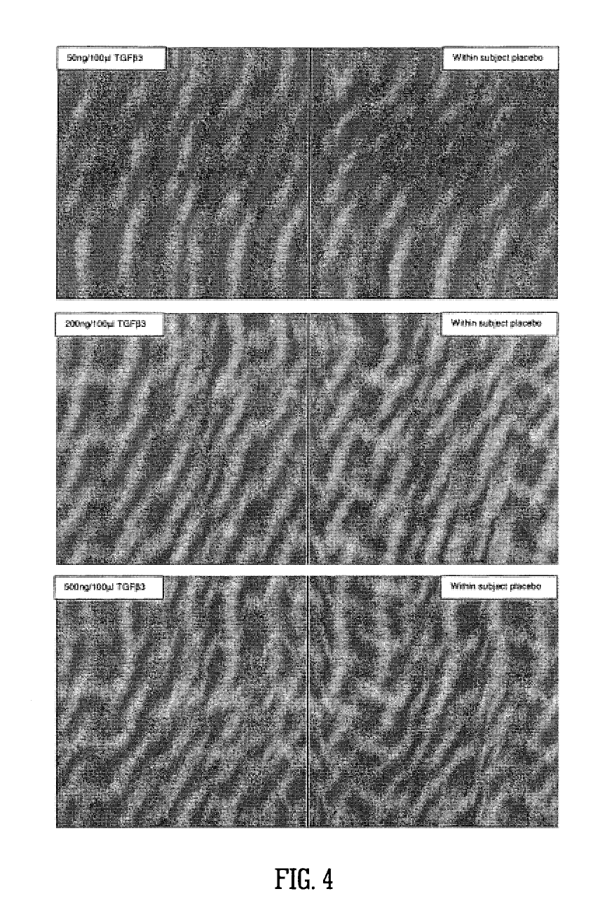

FIG. 4 compares macroscopic images of TGF-β3 control treated scars or placebo treated control scars. The three TGF-β3-treated scars were provided with different amounts of TGF-β3 in incidences of treatment separated by about 24 hours.

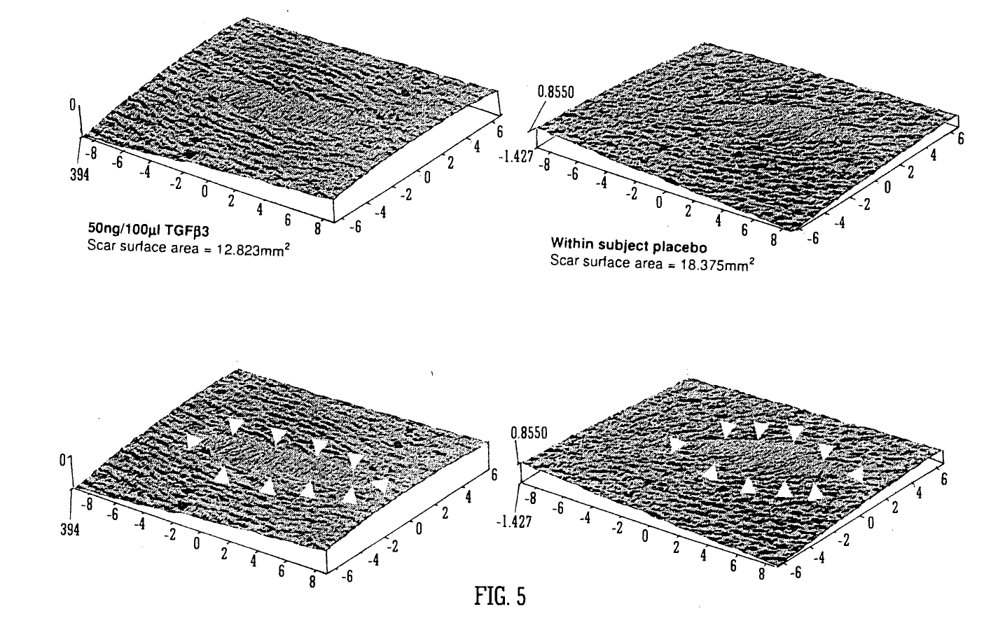

FIG. 5 illustrates 3-dimensional simulations and scar measurements taken from scars formed on healing of wounds treated with either TGF-β3 controls or placebo.

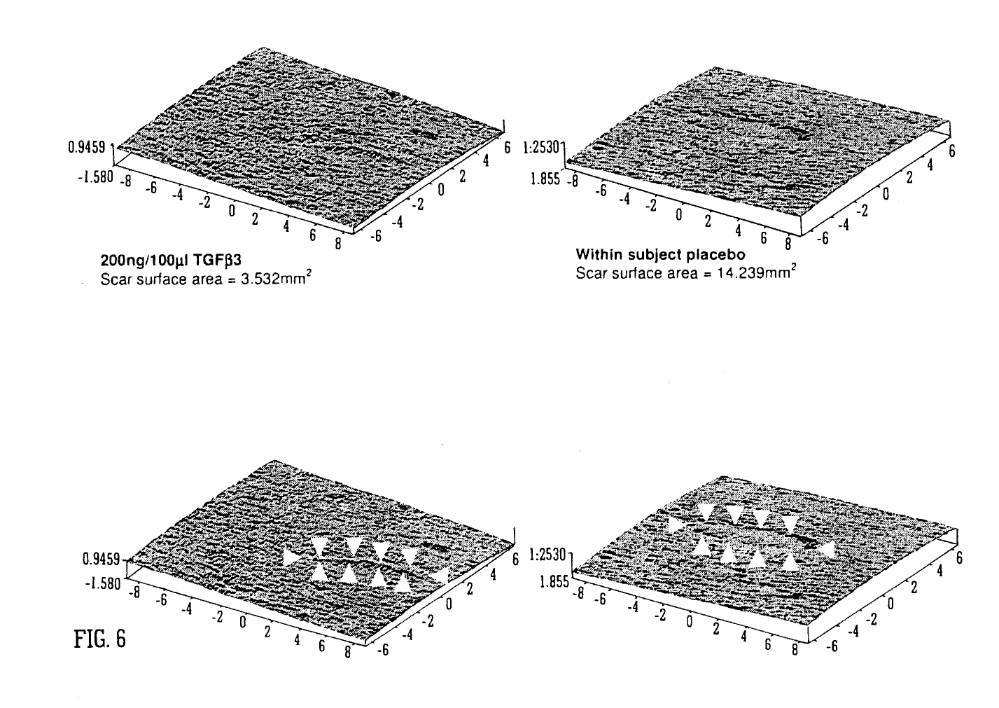

FIG. 6 illustrates 3-dimensional simulations and scar measurements taken from scars formed on healing of wounds treated with either TGF-β3 controls or placebo.

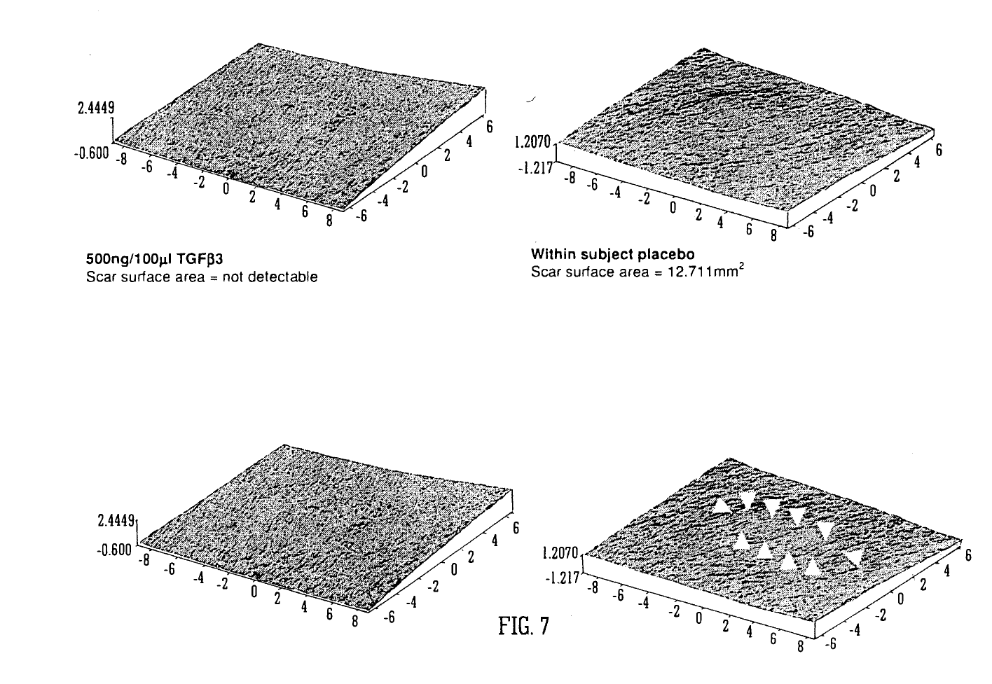

FIG. 7 illustrates 3-dimensional simulations and scar measurements taken from scars formed on healing of wounds treated with either TGF-β3 or with placebo.

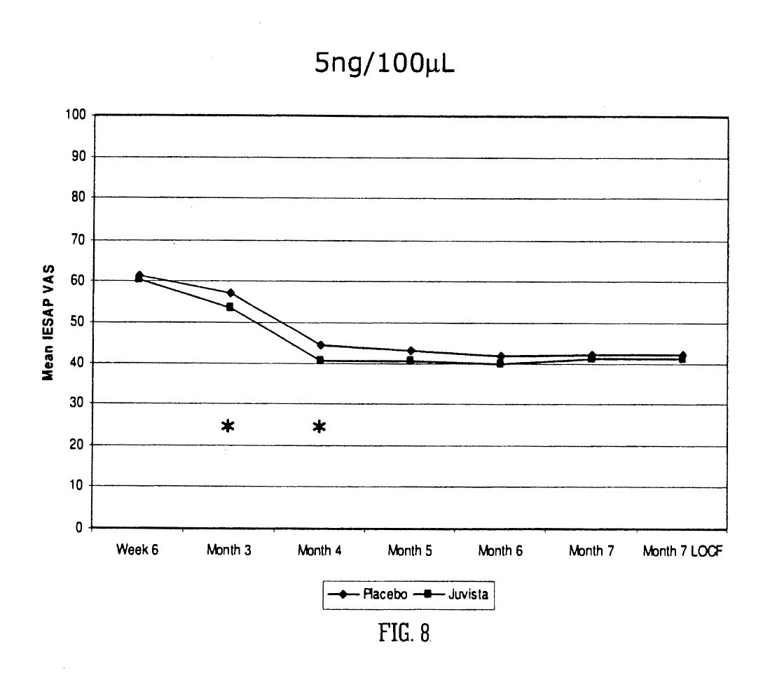

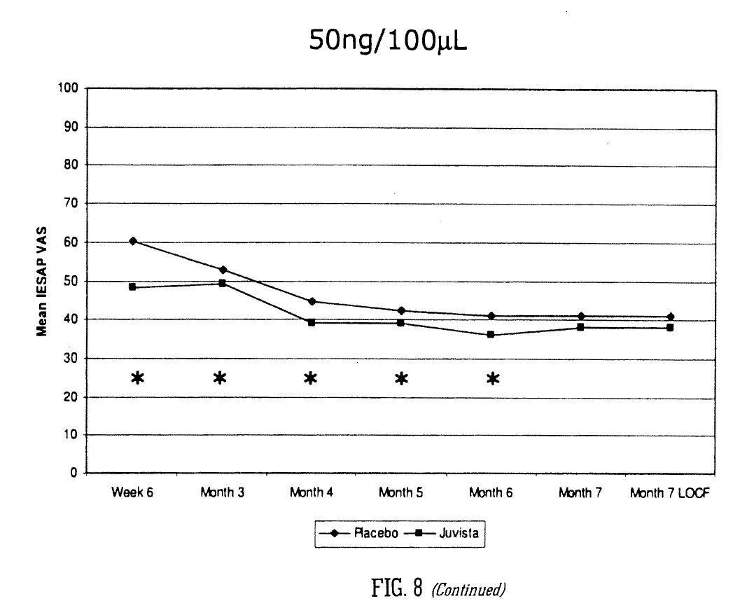

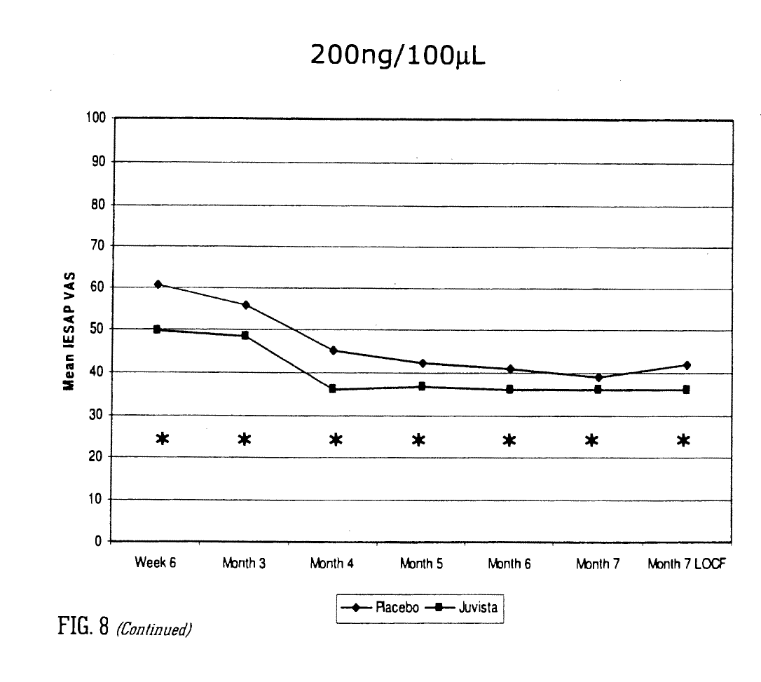

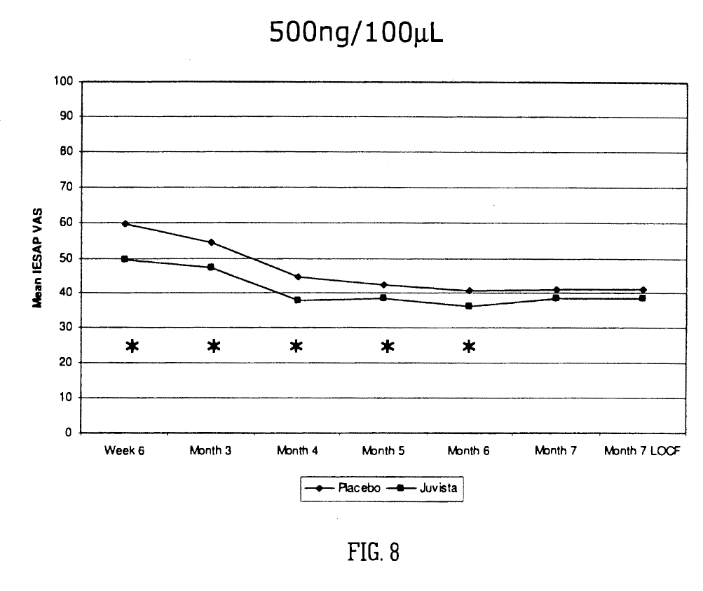

FIG. 8 compares the magnitude of inhibition of scarring achieved over time in control treated scars formed on healing of wounds treated with one of four experimental regimes using TGF-β3 (administered in an amount of 5 ng, 50 ng, 200 ng or 500 ng per centimetre in each of two incidences of treatment separated by approximately one hour).

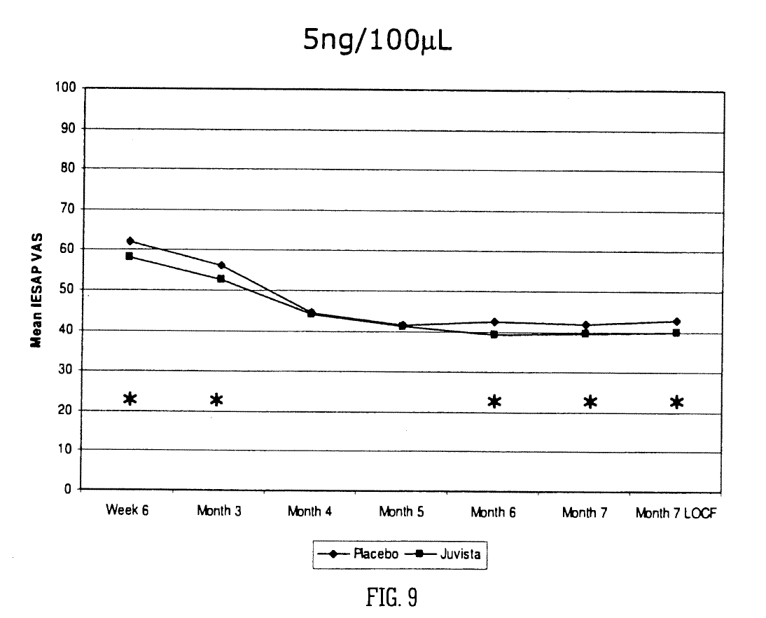

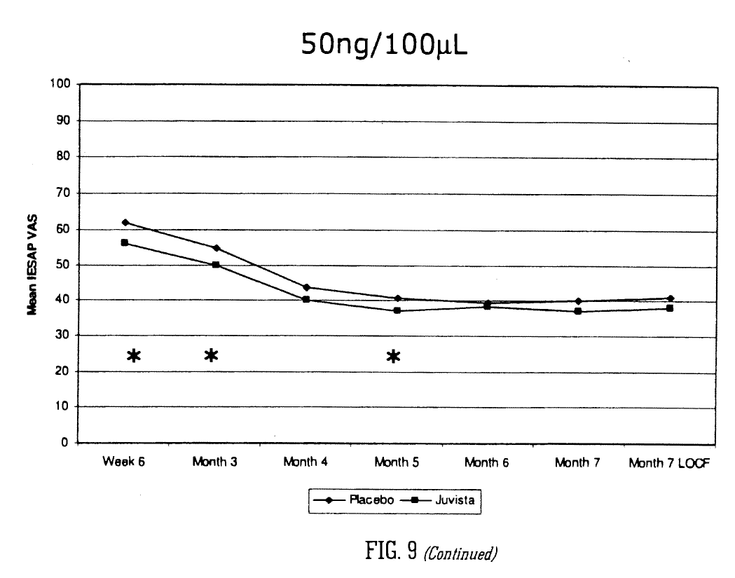

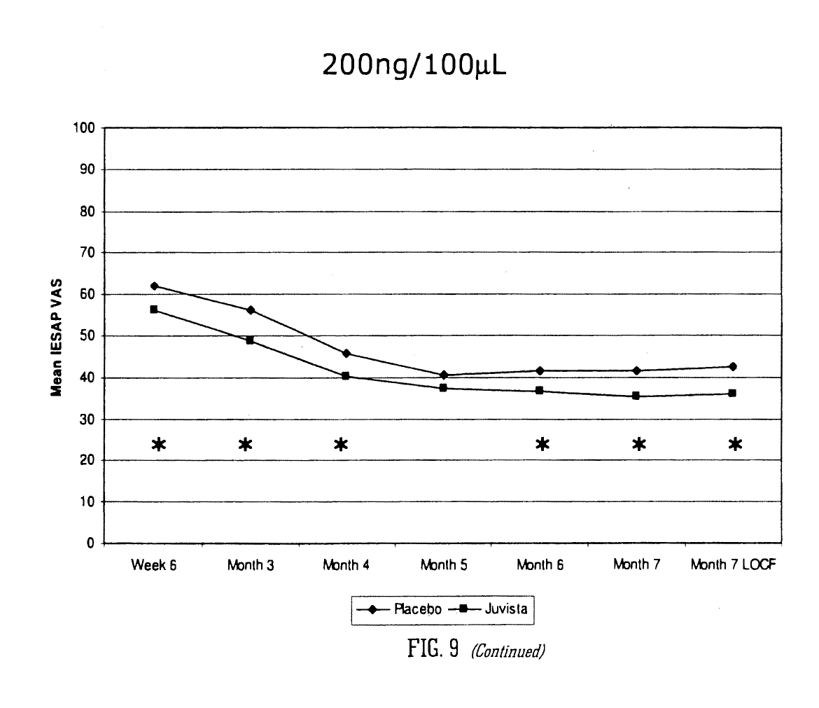

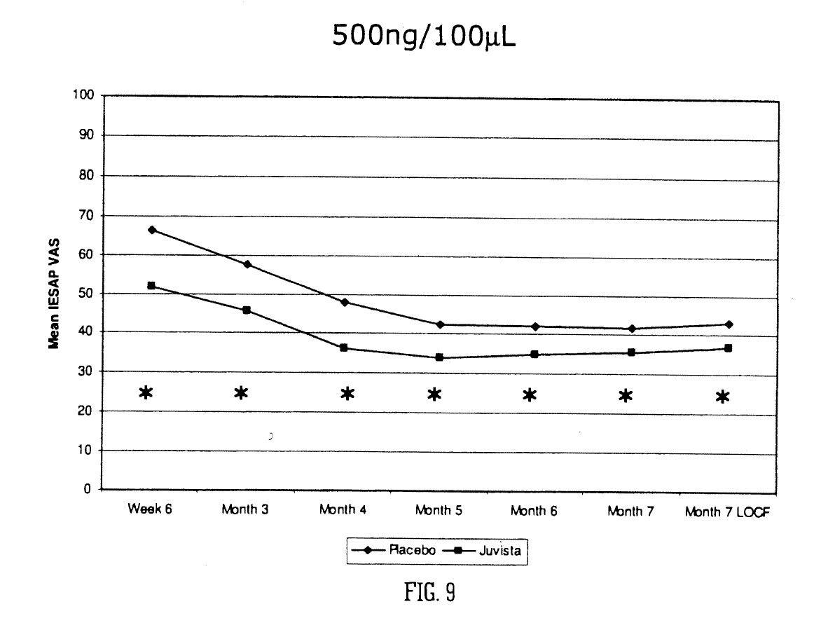

FIG. 9 compares the magnitude of inhibition of scarring achieved over time in control treated scars formed on healing of wounds treated with one of four experimental regimes using TGF-β3 (administered in an amount of 5 ng, 50 ng, 200 ng or 500 ng per centimetre in each of two incidences of treatment separated by approximately 24 hours).

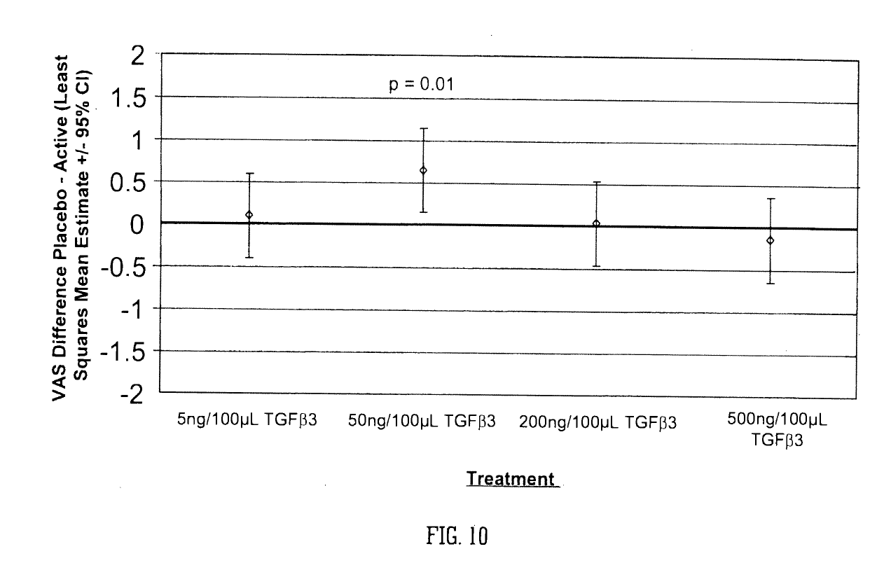

FIG. 10 illustrates a “bell-shaped” dose response curve in a rat model of scar formation in response to different doses of TGF-β3. TGF-β3 was provided to wounds via two injections of TGF-β3 separated by approximately 24 hours. The amount of TGF-β3 provided in each injection was the same in each incidence of treatment.

FIG. 11 compares the magnitude of inhibition of scarring achieved on healing of control treated wounds (each subject to two incidences of treatment, in which the amount of TGF-β3 administered remains constant between incidences of treatment) and on healing of wounds treated in accordance with the present invention.

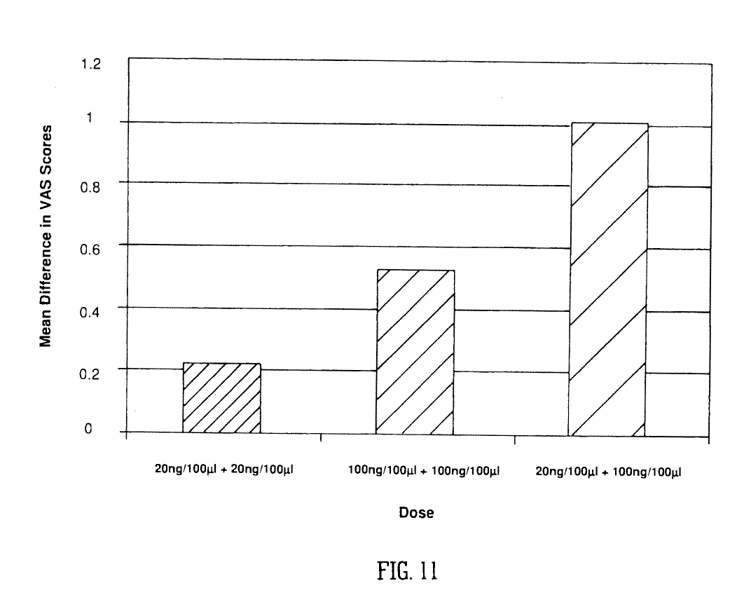

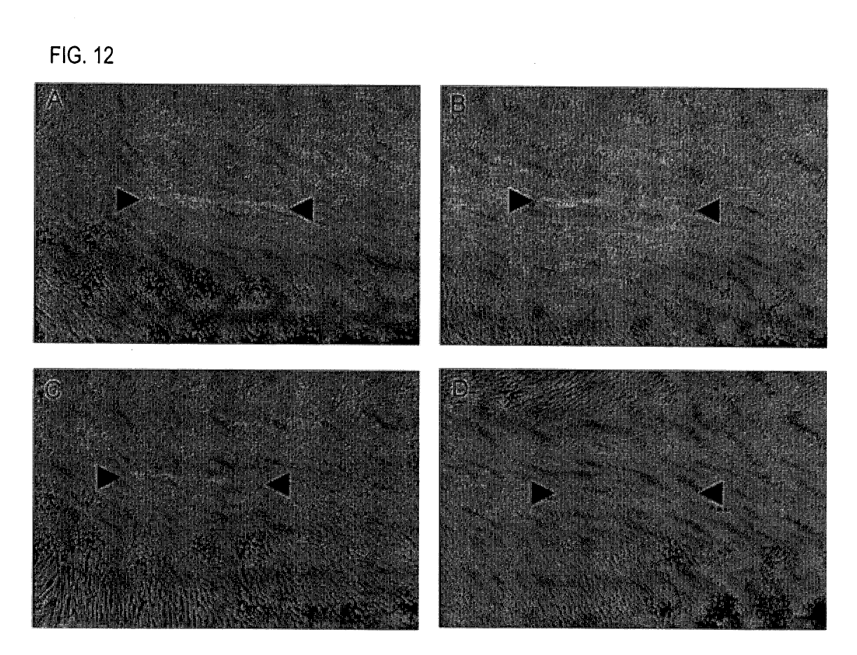

FIG. 12 shows representative images of scars produced on healing of placebo treated wounds (provided with diluent control in two incidences of treatment), control treated wounds (each subject to two incidences of treatment, in which the amount of TGF-β3 administered remains constant between incidences of treatment) and scars produced on healing of wounds treated in accordance with the present invention.

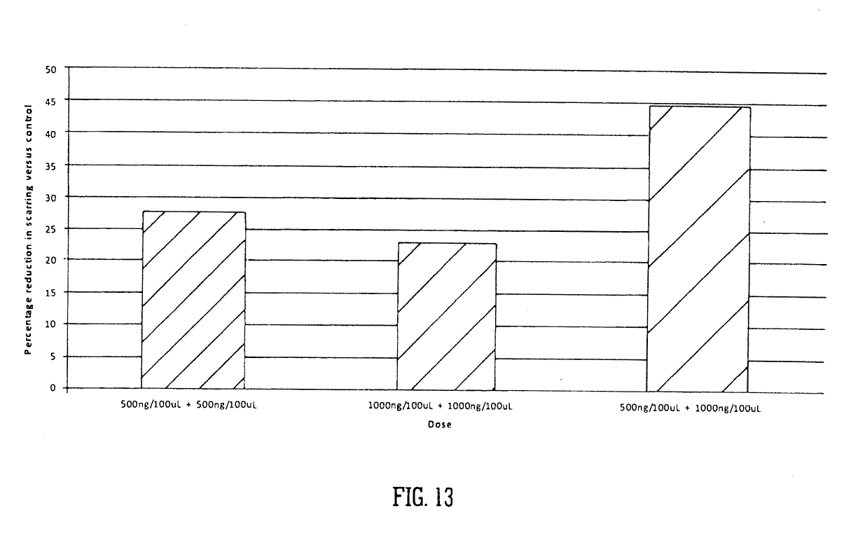

FIG. 13 is a graph comparing the percentage reduction in scarring achieved using methods of the invention employing the anti-scarring agent IL-10 with the reduction in scarring using the same agent in control treatment regimes.

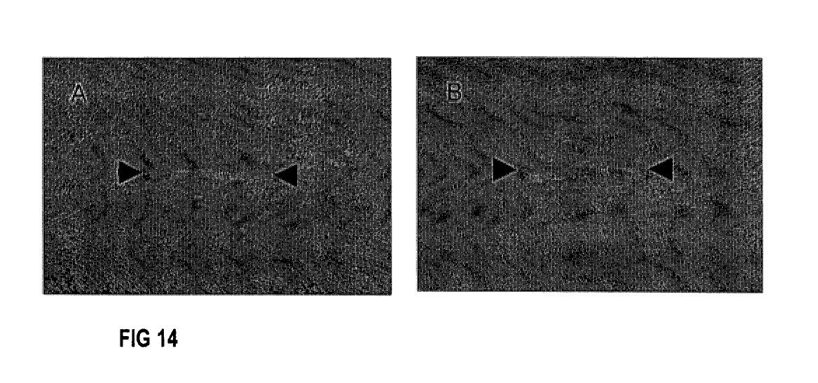

FIG. 14 illustrates macroscopic images of scars produced on healing of wounds treated using the anti-scarring agent IL-10 in accordance with the invention (Panel B) with scarring produced on healing of wounds treated with the same anti-scarring agent in control regimes (Panel A).

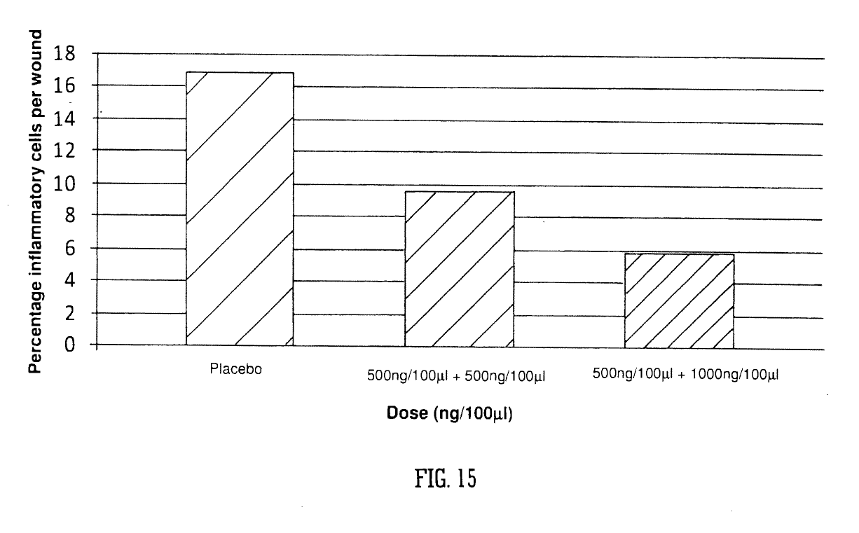

FIG. 15 compares the percentage of inflammatory cells found in wounds treated with the anti-scarring agent IL-10 in accordance with the present invention, and wounds treated with a placebo control, or control treatment using IL-10.

FIG. 16 shows photographs illustrating preferred routes of administration that may be used to provide an anti-scarring agent to a body site at which it is wished to inhibit scarring in accordance with the present invention. Panel A shows administration of a single injection of a composition comprising an anti-scarring agent at a site to be wounded. This injection has raised a bleb that covers the site where the wound will be formed (between the two inner dots) and covers an area that extends beyond the intended wound site (the area bounded by the outer dots). Panel B shows the administration of a composition comprising an anti-scarring agent along a future wound margin. The solid line illustrates the site where a wound is to be formed, and sites at which the anti-scarring agent may be administered are shown by the dots that surround the future wound. Panels C and D illustrate administration of compositions comprising an anti-scarring agent to the margins of existing wounds (which have been closed with sutures).

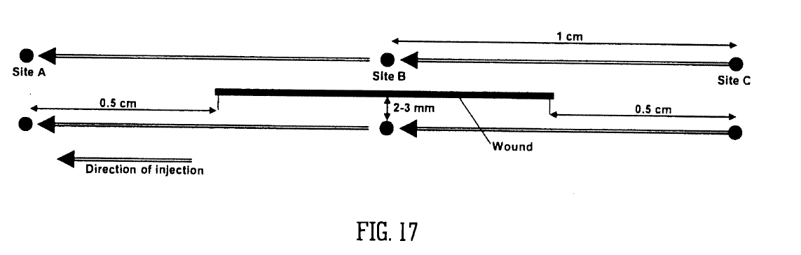

FIG. 17 illustrates a preferred method by which intradermal injections may be used for the administration of an anti-scarring agent in accordance with the present invention. A hypodermic needle through which the anti-scarring agent is to be administered is inserted intradermally at site B and advanced to site A (separated from site B by a distance of 1 cm). 100 μl of the composition is then administered evenly between sites A and B as the needle is withdrawn. The needle is then inserted intradermally at site C, advanced in the direction of site B, and the dosing process repeated. When administration to one margin of the wound has been completed, administration may then be repeated on the other margin.

### EXPERIMENTAL RESULTS

The results described below illustrate the use of treatment regimes in which an anti-scarring agent is provided to a wound, or site where a wound is to be formed, in two incidences of treatment, and show the effectiveness of such regimes in which the time elapsing between incidences of treatment is between 8 and 48 hours. They also clearly illustrate the advantageous inhibition of scarring that may be achieved in accordance with the present invention, where the amount of the anti-scarring agent provided is increased in the second incidence of treatment as compared to the first.

FIG. 1

FIG. 1 illustrates data from a clinical trial conducted by the inventors to generate a dose response curve indicative of the anti-scarring effect achieved using various different doses of TGF-β3 administered in a single incidence of treatment. Either TGF-β3 or placebo were administered as a single intradermal injection to a 1 centimetre experimental wound. The figure displays the treatment effect with TGFβ3 as least square means and 95% confidence intervals from an analysis of variance (ANOVA) with site as a factor. To test the treatment effect, ToScar of the TGFβ3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3-7) using a 100 mm VAS line.

FIG. 1 illustrates that scarring is effectively inhibited by a single application of 50 ng, 200 ng or 500 ng/100 μl TGFβ3 per cm of wound margin. The level of improvement displays a typical bell-shaped dose-response curve with maximum improvement (average >50 mm scar improvement in TGFβ3 treated wounds) observed at the 200 ng/100 μl dose, with a reduction in drug efficacy towards the top of the dose range i.e. 500 ng/100 μl per cm of wound margin

FIG. 2

FIG. 2 illustrates data from a clinical trial conducted by the inventors. In this study TGFβ3 and Placebo were each administered in two separate incidences of treatment (by means of two intradermal injections). However, unlike the methods of the present invention, the first incidence of treatment took place immediately prior to wounding but the second incidence of treatment occurred immediately after wound closure, i.e., both doses being administered within approximately 1 hour of one another (the first ten to thirty minutes prior to wounding, and the second ten to thirty minutes post-wounding). The figure displays the treatment effect with TGFβ3 as least square means and 95% confidence intervals from an analysis of variance (ANOVA) with site as a factor. To test the treatment effect, ToScar of the TGFβ3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3-7) using a 100 mm VAS line.

FIG. 2 illustrates that scarring is effectively inhibited by two applications of 5 ng, 50 ng, 200 ng and 500 ng/100 μl TGFβ3 per cm of wound margin, prior to and immediately after wound closure (i.e. both doses within approximately 1 hour). The level of improvement displays a typical bell-shaped dose-response curve with maximum improvement (average >40 mm scar improvement in TGFβ3 treated wounds) observed at the 200 ng/100 μl dose, with a reduction in drug efficacy towards the top of the dose range i.e. 500 ng/100 μl per cm of wound margin. The degree of improvement and dose-response curve with TGFβ3 treatment given twice (within approximately 1 hour) is comparable to that for TGFβ3 given once (see FIG. 1), though over all the degree to which scarring is inhibited is slightly less than for the single administration regime. This illustrates that repeated administration of TGF-β3 (other than in the methods described in the present invention) does not lead to a greater inhibition of scarring, and if anything may somewhat diminish the anti-scarring efficacy of this compound.

FIG. 3

FIG. 3 shows comparative data generated by the inventors in a human study. In this study, control treatments using TGFβ3 and Placebo were administered in two incidences of treatment (each by intradermal injection), the first prior to wounding and the second approximately 24 hours after wounding. The figure displays the treatment effect with TGFβ3 as least square means and 95% confidence intervals from an analysis of variance (ANOVA) with site as a factor. To test the effect of control treatment with TGF-β3, ToScar of the TGFβ3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3-7) using a 100 mm VAS line.

FIG. 3 illustrates that scarring is effectively inhibited by two applications of 5 ng, 50 ng, 200 ng and 500 ng/100 μl TGFβ3 per cm of wound margin, prior to and at approximately 24 hours post-wounding. Of these experimental methods of treatment, the method in which 500 ng TGF-β3 is administered in two incidences of treatment separated by 24 hours is notably more effective than the others.

FIG. 4

FIG. 4 shows representative macroscopic images from three subjects illustrating the different extents to which scarring may be inhibited using different TGFβ3 treatment regimes. The macroscopic images are from within subject scars produced on healing of placebo treated and TGFβ3 control treated wounds (dosed twice with 50 ng, 200 ng or 500 ng/100 μl TGFβ3 per cm of wound margin in two incidences of treatment approximately 24 hours apart) in a clinical trial conducted by the inventors. The same amount of TGF-β3 was administered in each incidence of treatment, and the amounts used are shown in the captions (50 ng/100 μl TGFβ3 per cm of wound margin shown top left, with placebo from the same subject top right; 200 ng/100 μl TGFβ3 per cm of wound margin shown middle left, with placebo from the same subject middle right; and 500 ng/100 μl TGFβ3 per cm of wound margin shown bottom left, with placebo from the same subject bottom right).

The wound receiving control TGF-β3 treatment at the highest dose used (bottom left) can be seen to benefit from the greatest inhibition of scarring achieved.

FIG. 5

FIG. 5 shows 3-dimensional simulations and scar measurements obtained from profilometry analysis of silicone moulds from scars produced on healing of placebo treated and TGFβ3 control treated wounds (dosed twice with 100 μl of 50 ng/100 μl TGFβ3 or 100 μl placebo per cm of wound margin approximately 24 hours apart) in a clinical trial conducted by the inventors. Note that this is not a method of treatment in accordance with the invention, but (along with FIG. 6) serves to provide comparative data illustrating the surprising effectiveness of a method of treatment in accordance with the invention.

The top panel shows the original 3-dimensional simulations and for clarity the bottom panel illustrates the boundaries of the scars demarcated by white arrowheads, with the remaining area of the image being normal skin surrounding the scar. A range of quantitative parameters for each scar were analysed by profilometry and demonstrated a 30.21% reduction in scar surface area with TGFβ3 treatment compared to placebo (TGFβ3 treated wound scar surface area=12.823 mm²; placebo treated wound scar surface area=18.375 mm²).

FIG. 6

FIG. 6 shows 3-dimensional simulations and scar measurements obtained from profilometry analysis of silicone moulds from scars produced on healing of placebo treated and TGFβ3 control treated wounds (dosed twice with 100 μl of 200 ng/100 μl TGFβ3 or 100 μl placebo per cm of wound margin approximately 24 hours apart) in a clinical trial conducted by the inventors. As with the results shown in FIG. 6, this does not constitute a method of treatment in accordance with the invention, but instead serves to provide comparative data illustrating the surprising effectiveness of a method of treatment in accordance with the invention.

The top panel shows the original 3-dimensional simulations and for clarity the bottom panel illustrates the boundaries of the scars demarcated by white arrowheads, with the remaining area of the image being normal skin surrounding the scar. A range of quantitative parameters for each scar were analysed by profilometry and demonstrated a 75.19% reduction in scar surface area with TGFβ3 treatment compared to placebo (TGFβ3 treated wound scar surface area=3.532 mm²; placebo treated wound scar surface area=14.239 mm²). Profilometry analysis also demonstrated a reduction in scar raised volume with TGFβ3 treatment of 73.33% compared to placebo treatment (TGFβ3 treated wound scar raised volume=0.0008 mm³; placebo treated wound scar raised volume=0.003 mm³).

FIG. 7

FIG. 7 shows 3-dimensional simulations and scar measurements obtained from profilometry analysis of silicone moulds from scars produced on healing of placebo treated and TGFβ3 control treated wounds (dosed twice with 100 μl of 500 ng/100 μl TGFβ3 or 100 μl placebo per cm of wound margin in two incidences of treatment providing equal amounts of TGF-β3 approximately 24 hours apart from one another).

The top panel shows the original 3-dimensional simulations and for clarity the bottom panel illustrates the boundaries of the scars demarcated by white arrowheads, with the remaining area of the image being normal skin surrounding the scar. Maximal inhibition of scarring achieved in this study is observed in response to treatment with two relatively high doses of TGF-β3. While this approach may be effective to inhibit scarring, it will be appreciated that the cost associated with such treatment regimes will be higher than for methods of treatment in accordance with the present invention (where effective inhibition of scarring may be achieved while using a smaller overall quantity of an anti-scarring agent).

FIG. 8

FIG. 8 illustrates data from a clinical trial conducted by the inventors in which either TGF-β3 or placebo were administered in two incidents of treatment (each comprising administration of the test substance by intradermal injection), the first incidence occurring prior to wounding and the second immediately after wound closure, i.e., both doses of TGF-β3 being the same as one another, and administered within approximately 1 hour (10-30 mins prior to wounding and 10-30 mins post wounding). It will be recognised that the experimental methods of treatment, the results of which are shown in FIG. 8, do not represent methods of treatment in accordance with the present invention, but are instead alternative (therapeutically effective) methods of treatment that illustrate the surprising efficacy of the methods of the invention.

FIG. 8 displays the treatment effect with TGF-β3 (here labelled “Juvista”) and placebo as mean visual analogue scale (VAS) scores (mm) The scars were scored by an independent lay panel at 6 time points after dosing (week 6 and months 3-7) using a 100 mm VAS line.

FIG. 8 illustrates that scarring is inhibited by two applications of 100 μl of 5 ng, 50 ng, 200 ng and 500 ng/100 μl TGF-β3 per cm of wound margin administered prior to and immediately after wound closure (i.e. both doses within approximately 1 hour). The level of improvement is dose responsive and typically is first evident at early time points (week 6 onwards) and is maintained throughout the assessment period (i.e., up to 7 months in this study).

* indicates significant difference (p<0.05) between scarring resulting from healing of wounds provided with the TGF-β3 control treatment and those provided with placebo treatment

FIG. 9

FIG. 9 illustrates data from a further clinical trial conducted by the inventors comparing therapeutically effective anti-scarring treatments using TGF-β3.

TGFβ3 and Placebo were administered by means of intradermal injection in two incidences of treatment, the first prior to wounding and the second approximately 24 hours later. The amount of TGF-β3 provided did not alter between incidences of treatment, and hence this study does not constitute a method of treatment in accordance with the present invention. The figure displays the treatment effect with TGFβ3 (once more labelled “Juvista”) and placebo as mean visual analogue scale (VAS) scores (mm). The scars were scored by an independent lay panel at 6 time points after dosing (week 6, months 3-7) using a 100 mm VAS line.

FIG. 9 illustrates that scarring is inhibited by two applications of 100 μl of 5 ng, 50 ng, 200 ng or 500 ng/100 μl TGFβ3 per cm of wound margin administered prior to wounding and at approximately 24 hour post-wounding. The level of improvement is dose responsive and typically is first evident at early time points (week 6 onwards) and is maintained throughout the assessment period (i.e., up to 7 months in this study). Surprisingly the magnitude of effect is much larger than expected from previous data. It can be seen that the method of the invention (in which 500 ng of TGF-β3 is provided per centimetre of the body site treated in each incidence of treatment) is surprisingly more effective than the other methods of treatment (which are themselves still therapeutically effective).

* indicates significant difference (p<0.05) between scarring resulting from healing of wounds provided with the TGFβ3 control treatment and those provided with Placebo treatment.

FIG. 10

FIG. 10 illustrates that the TGF-β3 “bell-shaped” dose response curve observed in human subjects is also found in experimental animals. Here, TGF-β3 was provided to experimental rat wounds, in two incidences of treatment separated by 24 hours (the first incidence of treatment occurring at, or around, the time of wounding). The amount of TGF-β3 administered per centimetre of wound in each incidence of treatment is shown on the X-axis (5 ng/cm, 50 ng/cm, 200 ng/cm or 500 ng/cm).

As can be seen, repeated treatment with low doses of TGF-β3 or with high doses of TGF-β3 brought about little inhibition of scarring.

FIG. 11

A rat experimental model of wound healing and scarring was used to illustrate the inhibition of scarring that may be achieved using escalating doses of TGF-β3 administered in sequential incidences of treatment, as compared to untreated controls, or control treatments with TGF-β3 in which the amount of TGF-β3 administered does not increase between first and second incidences of treatment.

FIG. 11 is a graph comparing the mean differences between macroscopic VAS scores of scars formed on healing of 1 cm incisional rat wounds treated with a diluent control (“placebo treated wounds”), and scars formed on healing of wounds provided with one of the following regimes:
i) TGF-β3 control treatment using 20 ng TGF-β3 per centimetre;
ii) TGF-β3 control treatment using 100 ng TGF-β3 per centimetre; or
iii) TGFβ3 treatment using escalating doses of TGF-β3 administered in sequential incidences of treatment.

In each case the wounds were subject to two incidences of treatment, the first prior to wounding and the second approximately 24 hours later.

Placebo treated control wounds were provided with two incidences of treatment, each of which consisted of administration of a diluent. These placebo treated wounds provide a baseline value for scarring, with reference to which scar inhibition produced by TGF-β3 treatments may be determined. “Control treated wounds” were provided with two incidences of treatment, each comprising injections of TGF-β3 at either 20 ng/100 μl or 100 ng/100 μl (the same concentration of TGF-β3 being injected in each incidence of treatment). The “treated wounds” were provided with an escalating dose regimen in which the first incidence of treatment comprised an injection of 20 ng/100 μl TGFβ3, while the second incidence of treatment comprised an injection of 100 ng/100 μl TGFβ3.

Each animal received two wounds, and these were arranged so that the wounds of each animal included placebo treated wounds, as well as either treated wounds (examples treated using escalating doses of TGF-β3 administered in sequential incidences of treatment), or control treated wounds (receiving control treatment with TGF-β3 at the same dose in each incidence of treatment). This permits comparison between scars formed on healing of placebo treated wounds and treated or control treated wounds within the same subject. This study design allows intra-subject variability to be reduced when assessing the anti-scarring effect of TGFβ3 treatment (either control treatment or using escalating doses of TGF-β3 administered in sequential incidences of treatment).

Scars were assessed, and VAS scores produced, 70 days after wounding.

In keeping with the results reported in FIG. 10 above, control treated wounds (dosed twice with either 20 ng/100 μl or 100 ng/100 μTGFβ3) displayed a reduction in scarring as compared to control untreated wounds receiving placebo. This is not surprising, since the amounts of TGF-β3 are in the region shown to be most effective in the “bell-shaped” distribution in this model. However, it is a surprising finding that wounds in which a larger amount of TGFβ3 is provided in the second incidence of treatment than the therapeutically effective amount administered in the first incidence of treatment displayed a much larger magnitude of effect in terms of the inhibition of scarring achieved on healing of the wound. The anti-scarring effect of dosing with 20 ng/100 μl TGFβ3 followed by 100 ng/100 μl TGFβ3 is a much larger synergistic effect than that which would be expected by an additive anti-scarring effect achieved in line with the results of either 20 ng/100 μl or 100 ng/100 μl TGFβ3 dosed twice.

The results illustrate that the inhibition of scarring observed on healing of wounds treated using escalating doses of TGF-β3 administered in sequential incidences of treatment is much greater than that observed on healing of wounds treated using alternative treatment regimens involving the administration of TGF-β3 in two incidences of treatment providing equal doses of TGF-β3.

FIG. 12

FIG. 12 shows representative images of the macroscopic appearance of scars produced by the studies described in connection with FIG. 11 above. These images of the scars were collected 70 days post wounding, and the arrow heads shown mark the ends of the scars.

The scars shown are those formed on healing of 1 cm incisional rat wounds provided with two incidents of treatment, 24 hours apart, with either placebo (to provide placebo treated control wounds) or TGF-β3 (to produce either treated wounds, using escalating doses of TGF-β3 administered in sequential incidences of treatment, or control treated wounds).

Representative images of scars produced on the healing of control placebo treated wounds are shown in Panel A. Panel B illustrates scars produced on healing of TGFβ3 control treated wounds provided with two incidents of treatment, each comprising injection of 20 ng/100 μl TGFβ3. Panel C illustrates scars produced on healing of TGFβ3 control treated wounds provided with two incidents of treatment, each comprising injection of 100 ng/100 μl TGFβ3. The scars shown in Panel D were produced on healing of wounds treated using escalating doses of TGF-β3 administered in sequential incidences of treatment. In a first incidence of treatment they were injected with 20 ng/100 μl TGFβ3, and in a second incidence of treatment were injected with 100 ng/100 μl TGFβ3.

The images illustrate that scars resulting from wounds treated with TGFβ3 are reduced in comparison to placebo treated wounds, in that they exhibit reduced width, are less white (a reduction in hypopigmentation) and blend better with the surrounding skin. The fact that the control TGF-β3 treated wounds exhibit a reduction in scarring is consistent with the effects observed in the generation of the dose response curve shown above. As reported in connection with FIG. 11, the wounds treated with an escalating dose regimen of 20 ng/100 μl TGFβ3 prior to wounding followed by an injection of 100 ng/100 μl TGFβ3 approximately 24 hours later, display the greatest inhibition in scarring, with resultant scars which more closely approximate the surrounding unwounded skin than do scars produced on the healing of wounds treated with other treatment regimens.

FIG. 13

FIG. 13 is a graph showing the percentage reduction in scarring, as compared to placebo control, achieved in scars formed on healing of 1 cm incisional rat wounds treated with IL-10 administered by means of intradermal injection in two incidences of treatment, the first prior to wounding and the second approximately 24 hours later. Wounds dosed with IL-10 received either two injections of 500 ng/100 μl IL-10, two injections of 1000 ng/100 μl IL-10, or an escalating dose regime in accordance with the present invention, in which the first injection comprised 500 ng/100 μl IL-10 and the second injection comprised 1000 ng/100 μl IL-10.

FIG. 13 illustrates that scarring is effectively inhibited by either two applications of 500 ng/100 μl IL-10 or two applications of 1000 ng/100 μl IL-10 to wounds. The level of improvement is suggestive of a dose response curve with maximum improvement (27.5%) observed at the 500 ng/100 μl dose, and a reduction in drug efficacy towards the 1000 ng/100 μl range (22.8%). Surprisingly, wounds dosed in accordance with the methods of the invention (in which a larger amount of IL-10 is provided in the second incidence of treatment than the therapeutically effective amount administered in the first incidence of treatment) displayed a much larger magnitude of effect than observed with either 500 ng/100 μl or 1000 ng/100 μl dosed twice. The anti-scarring effect of dosing with 500 ng/100 μl IL-10 followed by 1000 ng/100 μl IL-10 is greater than that which would expected based on the reduction in scarring observed when wounds were dosed twice with 1000 ng/100 μl IL-10.

The results illustrate that the inhibition of scarring observed on healing of wounds treated with the methods of the invention is much greater than that observed on healing of wounds treated using alternative treatment regimens.

FIG. 14

FIG. 14 shows representative images of the macroscopic appearance of scars resulting from 1 cm incisional rat wounds dosed twice (24 hours apart) with 500 ng/100 μl IL-10 (A) or once with 500 ng/100 μl IL-10 followed by 1000 ng/100 μl IL-10 (B). Arrow heads mark the ends of the scars 70 days post wounding.

The images show that scars resulting from wounds treated with an escalating dose regimen of 500 ng/100 μl IL-10 prior to wounding followed by an injection of 1000 ng/100 μl IL-10 approximately 24 hours later, display a greater inhibition of scarring than wounds dosed twice with the same amount (500 ng/100 μl) of IL-10. The scars treated with the methods of the invention have reduced width, are less white (reduced hypopigmentation) and more closely approximate the surrounding unwounded skin than scars treated with other dosing regimens.

FIG. 15

FIG. 15 shows the percentage of inflammatory cells in 1 cm incisional rat wounds treated with two injections of either placebo, two injections of 500 ng/100 μl IL-10, or one injection of 500 ng/100 μl IL-10 followed by a second injection of 1000 ng/100 μl IL-10; wounds treated with IL-10 or placebo were on the same animal permitting within-subject comparison. Wounds were excised from the experimental rats at 3 days post treatment, fixed in 10% (v/v) buffered formal saline, processed for histology and stained with CD68 to assess inflammatory cell numbers.

FIG. 15 illustrates that IL-10 reduces infiltration of inflammatory cells into the wound when compared to controls. The wounds treated with the methods of the invention (escalating dose regimen) exhibit a surprisingly marked decrease in inflammatory cell numbers than that observed on healing of wounds treated using alternative treatment regimens (same dose IL-10 given twice).

Conclusion

The results presented above clearly indicate that using escalating doses of an anti-scarring agent administered in sequential incidences of treatment, are capable of increasing the extent to which scarring is inhibited beyond that which may be expected. This has been illustrated with reference to two separate biologically effective anti-scarring agents, the anti-scarring growth factors TGF-β3 and IL-10, indicating that the approach of using escalating doses of anti-scarring agents to successfully inhibit scarring may be applicable to a wide range of anti-scarring agents.

Sequence Information

TGF-β 3

(Sequence ID No. 1)

ALDTNYCFRNLEENCCVRPLYIDFRQDLGWKWVHEPKGYYANFCSGP
CPYLRSADTTHSTVLGLYNTLNPEASASPCCVPQDLEPLTILYYVGR
TPKVEQLSNMVVKSCKCS
Sequence ID No. 2 - DNA encoding wild-type
human TGF-β3
GCT TTG GAC ACC AAT TAC TGC TTC CGC AAC TTG GAG
GAG AAC TGC TGT GTG CGC CCC CTC TAC ATT GAC TTC
CGA CAG GAT CTG GGC TGG AAG TGG GTC CAT GAA CCT
AAG GGC TAC TAT GCC AAC TTC TGC TCA GGC CCT TGC
CCA TAC CTC CGC AGT GCA GAC ACA ACC CAC AGC ACG
GTG CTG GGA CTG TAC AAC ACT CTG AAC CCT GAA GCA
TCT GCC TCG CCT TGC TGC GTG CCC CAG GAC CTG GAG
CCC CTG ACC ATC CTG TAC TAT GTT GGG AGG ACC CCC
AAA GTG GAG CAG CTC TCC AAC ATG GTG GTG AAG TCT
TGT AAA TGT AGC
Interleukin 10 (IL-10)

(Sequence ID No. 3)

MSPGQGTQSE NSCTHFPGNL PNMLRDLRDA FSRVKTFFQ
MKDQLDNLLL KESLLEDFKG YLGCQALSEM IQFYLEEVMP
QAENQDPDI KAHVNSLGEN LKTLRLRLRR CHRFLPCENK
SKAVEQVKNA FNKLQEKGI YKAMSEFDIF INYIEAYMTM KIRN
DNA encoding Homo sapiens interleukin 10 (IL-10)

(Sequence ID No. 4)

ATG AGC CCA GGC CAG GGC ACC CAG TCT GAG AAC AGC
TGC ACC CAC TTC CCA GGC AAC CTG CCT AAC ATG CTT
CGA GAT CTC CGA GAT GCC TTC AGC AGA GTG AAG ACT
TTC TTT CAA ATG AAG GAT CAG CTG GAC AAC TTG TTG
TTA AAG GAG TCC TTG CTG GAG GAC TTT AAG GGT TAC
CTG GGT TGC CAA GCC TTG TCT GAG ATG ATC CAG TTT
TAC CTG GAG GAG GTG ATG CCC CAA GCT GAG AAC CAA
GAC CCA GAC ATC AAG GCG CAT GTG AAC TCC CTG GGG
GAG AAC CTG AAG ACC CTC AGG CTG AGG CTA CGG CGC
TGT CAT CGA TTT CTT CCC TGT GAA AAC AAG AGC AAG
GCC GTG GAG CAG GTG AAG AAT GCC TTT AAT AAG CTC
CAA GAG AAA GGC ATC TAC AAA GCC ATG AGT GAG TTT
GAC ATC TTC ATC AAC TAC ATA GAA GCC TAC ATG ACA
ATG AAG ATA CGA AAC TGA AAG

1. An anti-scarring agent for use as a medicament in treating a wound or site where a wound is to be formed to inhibit scarring, wherein in a first incidence of treatment the medicament is provided such that a first therapeutically effective amount of the anti-scarring agent is provided to each centimetre of a wound margin or each centimetre of a site at which a wound is to be formed; and wherein in a subsequent incidence of treatment the medicament is provided such that a larger therapeutically effective amount of the anti-scarring agent is provided to each centimetre of a wound margin between 8 hours and 48 hours after the previous incidence of treatment.

2. An anti-scarring agent according to claim 1, wherein the anti-scarring agent is an agent other than TGF-β3.

3. An anti-scarring agent according to claim 1 or claim 2, wherein the anti-scarring agent comprises interleukin-10 (IL-10), or a therapeutically effective fragment or derivative thereof.

4. An anti-scarring agent according to any preceding claim, wherein the use as a medicament further comprises a third or further incidence of treatment.

5. An anti-scarring agent according to claim 4, wherein the amount of the anti-scarring agent provided in a third or further incidence of treatment is substantially the same as the amount provided in the second incidence of treatment.

6. An anti-scarring agent according to any one of claims 1 to 4, wherein the therapeutically effective amount of the anti-scarring agent provided in a third or further incidence of treatment, is larger than the amount of the anti-scarring agent provided in the preceding incident of treatment.

7. An anti-scarring agent according to claim 6, wherein the amount of the anti-scarring agent provided per centimetre of wounding in the second, or further, incidence of treatment is at least 10% larger than the amount provided in the preceding incident of treatment.

8. An anti-scarring agent according to claim 7, wherein the amount of the anti-scarring agent provided per centimetre of wounding in the second, or further, incidence of treatment is at least 50% larger than the amount provided in the preceding incident of treatment.

9. An anti-scarring agent according to any preceding claim, wherein the incidences of treatment are separated by approximately 24 hours.

10. An anti-scarring agent according to any preceding claim, wherein the medicament is for use in the skin.

11. An anti-scarring agent according to any preceding claim, where the medicament is for use in the circulatory system

12. An anti-scarring agent according to any preceding claim, wherein the medicament is for use to inhibit scarring as a result of surgery.

13. An anti-scarring agent according to any preceding claim, wherein the medicament is for provision by local injection.

14. Interleukin-10 (IL-10), or a therapeutically effective fragment or derivative thereof, for use as a medicament in treating a wound or site where a wound is to be formed to inhibit scarring, wherein in a first incidence of treatment the medicament is provided such that a first therapeutically effective amount of the IL-10, or therapeutically effective fragment or derivative thereof, is provided to each centimetre of a wound margin or each centimetre of a site at which a wound is to be formed; and wherein in a subsequent incidence of treatment the medicament is provided such that a larger therapeutically effective amount of the IL-10, or therapeutically effective fragment or derivative thereof, is provided to each centimetre of a wound margin between 8 hours and 48 hours after the previous incidence of treatment.

15. A method of inhibiting scarring formed on healing of a wound, the method comprising treating a body site in which scarring is to be inhibited:
in a first incidence of treatment providing to each centimetre of wound margin, or each centimetre of a site at which a wound is to be formed a first therapeutically effective amount of an anti-scarring agent; and
in a second incidence of treatment, occurring after a wound is formed and between 8 and 48 hours after the first incidence of treatment, providing to said wound a therapeutically effective amount of said anti-scarring agent that is larger than the therapeutically effective amount of the anti-scarring agent provided in the first incidence of treatment.

16. The method according to claim 15, wherein the anti-scarring agent is provided by means of a local injection.

17. The method according to claim 16, wherein the first incidence of treatment is provided at a site where a wound is to be formed and the local injection is to be administered substantially along the midline of the wound to be formed.

18. The method according to claim 16, wherein the first incident of treatment is provided to a site at which a wound is to be formed and wherein a local injection is administered on each of the margins of the wound to be formed.

19. The method according to claim 16, wherein the first and or second incidence of treatment is provided to a wound margin and the local injection is administered at a location within half a centimetre of the wound margin

20. The method according to any one of claims 15 to 19, wherein the first and/or second incidence of treatment comprises providing the anti-scarring agent to a region extending at least half a centimetre beyond each end of the wound.

21. A method of inhibiting scarring formed on healing of a wound, the method comprising treating a body site in which scarring is to be inhibited:
in a first incidence of treatment providing to each centimetre of a site where a wound is to be formed a first therapeutically effective amount of an anti-scarring agent; and
in a second incidence of treatment, occurring after a wound is formed and between 8 and 48 hours after the first incidence of treatment, providing to said wound a therapeutically effective amount of said anti-scarring agent that is larger than the therapeutically effective amount of the anti-scarring agent provided in the first incidence of treatment.

22. A method of inhibiting scarring formed on healing of a wound, the method comprising treating a body site in which scarring is to be inhibited:
in a first incidence of treatment providing to each centimetre of wound margin, or each centimetre of future wound margin, a first therapeutically effective amount of an anti-scarring agent; and
in a second incidence of treatment, occurring after a wound is formed and between 8 and 48 hours after the first incidence of treatment, providing to said wound a therapeutically effective amount of said anti-scarring agent that is larger than the therapeutically effective amount of the anti-scarring agent provided in the first incidence of treatment.

23. A method according to any one of claims 15 to 22, further comprising a third or further incidence of treatment.

24. A method according to claim 23, wherein the amount of the anti-scarring agent provided in the third or further incidence of treatment is substantially the same as the amount provided in the second incidence of treatment.

25. A method according to any one of claims 15 to 23, wherein the therapeutically effective amount of the anti-scarring agent provided in the third or further incidence of treatment, is larger than the amount of the anti-scarring agent provided in the preceding incident of treatment.

26. A method according to claim 25, wherein the amount of the anti-scarring agent provided per centimetre of wounding in the second, or further, incidence of treatment is at least 10% larger than the amount provided in the preceding incident of treatment.

27. A method according to claim 26, wherein the amount of the anti-scarring agent provided per centimetre of wounding in the second, or further, incidence of treatment is at least 50% larger than the amount provided in the preceding incident of treatment.

28. A method according to any one of claims 15 to 27, wherein the incidences of treatment are separated by approximately 24 hours.

29. A method according to any one of claims 15 to 28, wherein the wound is a skin wound.

30. A method according to any one of claims 15 to 29, where the wound is a wound of the circulatory system

31. A method according any one of claims 15 to 30, wherein the wound is a result of surgery.

32. A method according to any one of claims 15 to 31, wherein the anti-scarring agent is provided by local injection administered to the body site.

33. A method according to any one of claims 15 to 32, wherein the anti-scarring agent is provided in a pharmaceutically acceptable solution, approximately 100 μl of which is administered per centimetre of body site treated.

34. A method according to any one of claims 21 to 33, wherein the first incidence of treatment occurs prior to wounding.

35. A method according to claim 34, wherein the first incidence of treatment occurs up to an hour prior to wounding.

36. A method according to any one of claims 21 to 33, wherein the first incidence of treatment occurs after wounding.

37. A method according to claim 36, wherein the first incidence of treatment occurs up to two hours after wounding.

38. A method according to any one of claims 21 to 33, wherein the first incidence of treatment occurs after wound closure.

39. A method according to claim 38, wherein the first incidence of treatment occurs up to two hours after wound closure.

40. A method of selecting an appropriate treatment regime for inhibiting scarring associated with the healing of a wound, the method comprising:
determining whether an individual in need of such inhibition of scarring will be able to complete a second incidence of treatment occurring between 8 and 48 hours after a first incidence of treatment;
if the individual will be able to complete a second incidence of treatment occurring between 8 and 48 hours after a first incidence of treatment, selecting a treatment regime comprising treating a body site in which scarring is to be inhibited such that:
in a first incidence of treatment providing to each centimetre of wound margin, or each centimetre of a site at which a wound is to be formed a first therapeutically effective amount of an anti-scarring agent; and
in a second incidence of treatment, occurring after a wound is formed and between 8 and 48 hours after the first incidence of treatment, providing to said wound a therapeutically effective amount of said anti-scarring agent that is larger than the therapeutically effective amount of the anti-scarring agent provided in the first incidence of treatment; or
if the individual will not be able to complete a second incidence of treatment occurring between 8 and 48 hours after a first incidence of treatment, selecting a treatment regime comprising:
in a single incidence of treatment providing to each centimetre of wound margin, or each centimetre of a site at which a wound is to be formed, in which scarring is to be inhibited an amount of the anti-scarring agent that is known to be inhibit scarring in a single incidence of treatment.

41. A kit for use in the inhibition of scarring associated with healing of a wound, the kit comprising at least first and second vials comprising an anti-scarring agent for administration to a wound, or a site where a wound is to be formed, at times between 8 and 48 hours apart from one another.

42. A kit for use in the inhibition of scarring associated with healing of a wound, the kit comprising:
a first amount of a composition containing an anti-scarring agent, this first amount being for administration to a wound, or a site where a wound is to be formed, in a first incidence of treatment;
a second amount of a composition containing the anti-scarring agent, this second amount being for administration to a wound in a second incidence of treatment;
instructions regarding administration of the first and second amounts of the composition at times between 8 and 48 hours apart from one another, and in a manner such that a larger therapeutically effective dose of the anti-scarring agent is administered to the wound in the second incidence of treatment than was administered in the first incidence of treatment.

43. A kit according to claim 41 or 42, comprising IL-10, or a therapeutically effective fragment or derivative thereof, as an anti-scarring agent.

44. A kit according to claim 42 or 43, wherein the first and second amounts of a composition respectively comprise different first and second compositions, wherein the second composition contains the anti-scarring agent at a greater concentration than does the first composition

45. A kit according to claim 42, wherein the first and second compositions contain the anti-scarring agent at substantially equal concentrations and the instructions indicate that the volume of the second composition administered in the second incidence of treatment should be larger than the volume of the first composition administered in the first incidence of treatment.


SEQUENCE LISTING (4 sequences)

SEQ ID NO: 1 - protein, 112 aa, Homo sapiens [sequence omitted]

SEQ ID NO: 2 - DNA, 336 bp, Homo sapiens
gctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagc

SEQ ID NO: 3 - protein, 161 aa, Homo sapiens
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - DNA, 489 bp, Homo sapiens
atgagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgaaag